(19) United States
(12) Patent Application Publication
(10) Pub. No.: US 2022/0098586 A1
(43) Pub. Date: Mar. 31, 2022

(54) OLIGONUCLEOTIDE

(71) Applicant: BioMarin Technologies B.V., Leiden (NL)

(72) Inventors: Peter Christian de Visser, Leiden (NL); Judith Christina Theodora van Deutekom, Dordrecht (NL)

(21) Appl. No.: 17/449,290

(22) Filed: Sep. 29, 2021

Related U.S. Application Data
(60) Provisional application No. 63/085,668, filed on Sep. 30, 2020.

(51) Int. Cl. C12N 15/113; A61P 21/00

(52) U.S. Cl. CPC C12N 15/113; A61P 21/00; C12N 2310/321; C12N 2310/3341; C12N 2310/315; C12N 2310/3231; C12N 2310/11

(57) ABSTRACT

Provided are splice-switching hydroxyalkoxylated antisense oligonucleotides for preventing, treating, and/or delaying neuromuscular disorders, more specifically Duchenne Muscular Dystrophy.

### RELATED APPLICATIONS

This application claims benefit of priority to U.S. Provisional Application No. 63/085,668, filed Sep. 30, 2020, the contents of which are incorporated by reference herein in their entirety.

### SEQUENCE LISTING

This application contains a Sequence Listing, which is being submitted herewith as an ASCII text file named “0105_26_US_SL.txt”, created on Sep. 20, 2021, size 15,293,355 bytes, which is incorporated by reference herein in its entirety.

### FIELD

Provided are hydroxyalkoxylated antisense oligonucleotides, more specifically splice-switching hydroxyalkoxylated antisense oligonucleotides for the treatment of genetic disorders, more specifically neuromuscular disorders. Also provided are methods of using the hydroxyalkoxylated antisense oligonucleotides for treating, preventing and/or delaying Duchenne Muscular Dystrophy (DMD).

### BACKGROUND

Antisense oligonucleotides (AONs) are in (pre)clinical development for many diseases and conditions, including cancer, inflammatory conditions, cardiovascular disease and neurodegenerative and neuromuscular disorders. Their mechanism of action is aimed at various targets, such as RNaseH-mediated degradation of target RNA in the nucleus or cytoplasm, at splice-modulation (exon inclusion or skipping) in the nucleus, or at translation inhibition by steric hindrance of ribosomal subunit binding in the cytoplasm. Splice-modulating or splice-switching oligonucleotides (SSOs) were first described for correction of aberrant splicing in human β-globin pre-mRNAs (Dominski and Kole PNAS, 1993, 90(18):8673-8677), and are currently being studied for a variety of genetic disorders including, but not limited to, cystic fibrosis (CFTR gene, Friedman et al., J Biol Chem 1999, 274(51):36193-36199), breast cancer (BRCA1 gene, Uchikawa et al., J Hum Genet 2007, 52(11):891-897), prostate cancer (FOLH1 gene, Williams et al., Oligonucleotides 2006, 16(2):186-95), inflammatory diseases (IL-5Ralpha and MyD88 genes, Karras et al., Biochemistry 2001, 40(26):7853-9, Vickers et al., J Immunol 2006, 176(6):3652-61), ocular albinism type 1 (OA1 gene, Vetrini et al., Hum Mutat 2006, 27(5):420-6), ataxia telangiectasia (ATM gene, Du et al., PNAS 2007, 104(14):6007-12), nevoid basal cell carcinoma syndrome (PTCH1 gene, J Invest Dermatol 2006, 126(12):2614-20 et al., J Hum Genet 2007, 52(11):891-897), methylmalonic acidemia (MUT gene, Rincon et al., Am J Hum Genet 2007, 81(6):1262-1270), preterm labor (COX-2 gene, Tyson-Capper et al., Mol Pharmacol 2006, 69(3):796-804), artherosclerosis (APOB gene, Khoo et al., BMC Mol Biol 2007, 8; 3), propionic acidemia (PCCA, PCCB genes, Rincon et al., Am J Hum Genet 2007, 81(6):1262-1270), leukemia (c-myc and WT1 genes, Renshaw et al., Mol Cancer Ther 2004, 3(11):1467-84, Giles et al., Antisense Nucleic Acid Drug Dev 1999, 9(2):213-20), dystrophic epidermolysis bullosa (COL7A1 gene, Goto et al., 2006), familial hypercholesterolemia (APOB gene, Disterer et al., Mol Ther 2013, 21(3):602-609), laser-induced choroidal neovascularization and corneal graft rejection (KDR gene, Uehara et al., FASEB J 2013, 27(1):76-85), hypertrophic cardiomyopathy (MYBPC3 gene, Gedicke-Hornung et al., EMBO Mol Med 2013, 5(7):1060-77), Usher syndrome (USH1C gene, Lentz et al., Nat Med 2013, 19(3):345-350), fukuyama congenital muscular dystrophy (FKTN gene, Taniguchi-Ikeda et al., Nature 2011, 478(7367):127-31), laser-induced choroidal neovascularization (FLT1 gene, Owen et al., PLoS One 2012, 7(3):e33576), cancer (STAT3 and bcl-X genes, Zammarchi et al., PNAS 2011, 108(43):17779-84, Mercatante et al., J Biol Chem 2002, 277(51):49374-82), and Hutchinson-Gilford progeria (LMNA gene, Osorio et al., Sci Transl Med 2011, 3(106):106ra107), Miyoshi myopathy (DYSF gene, Wein et al., Hum Mut 2010, 31(2):136-42), spinocerebellar ataxia type 1 (ATXN1 gene, Gao et al., Cell Transplant 2008, 17(7):723-34), Alzheimer's disease/FTDP-17 taupathies (MAPT gene, Peacey et al., NAR 2012, 40(19):9836-49), myotonic dystrophy (CLC1 gene, Wheeler et al., J Clin Invest 2007, 117(12):3952-7), and Huntington's disease (Evers et al., Nucleic Acid Ther 2014, 24(1):4-12). However, splice-switching AONs have progressed furthest in the treatment of the neuromuscular disorders Duchenne muscular dystrophy (DMD) and Becker muscular dsytrophy (BMD).

Duchenne muscular dystrophy (DMD) and Becker muscular dystrophy (BMD) are the most common childhood forms of muscular dystrophy. DMD is a severe, lethal neuromuscular disorder resulting in a dependency on wheelchair support before the age of 12 and patients often die before the age of thirty due to respiratory or heart failure. It is caused by reading frame-shifting deletions (˜67%) or duplications (˜7%) of one or more exons, or by point mutations (˜25%) in the 2.24 Mb dystrophin gene, resulting in the absence of functional dystrophin. BMD is also caused by mutations in the dystrophin gene, but these maintain the open reading frame, yield semi-functional dystrophin proteins, and result in a typically much milder phenotype and longer lifespan. During the last decade, specific modification of splicing in order to restore the disrupted reading frame of the transcript has emerged as a promising therapy for DMD (van Ommen et al., Curr Opin Mol Ther. 2008; 10(2):140-9; Yokota et al., Acta Myol. 2007; 26(3):179-84; van Deutekom et al., N Engl J Med. 2007; 357(26):2677-86; Goemans et al., N Engl J Med. 2011; 364(16):1513-22; Voit et al., Lancet Neurol 2014, 13(10):987-96; Cirak et al., Lancet 2011; 378: 595-605). Using highly sequence-specific splice-switching antisense oligonucleotides (AONs) which bind to the exon flanking or containing the mutation and which interfere with its splicing signals, the skipping of that exon can be induced during the processing of the dystrophin pre-mRNA. Despite the resulting truncated transcript, the open reading frame is restored and a protein is produced which is similar to those found in BMD patients. AON-induced exon skipping provides a mutation-specific, and thus personalized, therapeutic approach for DMD patients. As the majority of the mutations cluster around exons 45 to 55, the skipping of one specific exon may be therapeutic for many patients with different mutations. The skipping of exon 51 applies to the largest subset of patients (˜13%), including those with deletions of exons 45 to 50, 48 to 50, 50, or 52. The AONs applied can be chemically modified to resist endonucleases, exonucleases, and RNaseH, and to promote RNA binding and duplex stability. Different AON chemistries are currently being explored for inducing corrective exon skipping for DMD, including 2′-O-methyl phosphorothioate RNA (2OMePS; Voit et al., Lancet Neurol 2014, 13(10):987-96), phosphorodiamidate morpholino (PMO; Cirak et al., Lancet 2011; 378: 595-605), tricyclo DNA (tcDNA; Goyenvalle et al, Nat Med 2015, 21(3):270-5), and peptide nucleic acid (PNA; Gao et al., Mol Ther Nucleic Acids 2015, 4:e255). Although AONs are typically not well taken up by healthy muscle fibers, the dystrophin deficiency in DMD and the resulting pathology, characterized by activated satellite cells and damaged and thus more permeable fiber membranes, actually facilitates a better uptake. In studies in the dystrophin-deficient mdx mouse model, 2′-O-methyl phosphorothioate RNA oligonucleotides have indeed demonstrated an up to 10 times higher uptake in different muscle groups when compared to that in wild type mice (Heemskerk et al., Mol Ther 2010; 18(6):1210-7). Clinical study results with both 2′-O-methyl phosphorothioate RNA and phosphorodiamidate morpholino AONs in DMD patients confirm presence of the AONs in muscle biopsies, but the levels of novel dystrophin after treatment were still limited, which challenges the field to develop oligonucleotides with improved characteristics enhancing therapeutic index and clinical applicability.

Clinical efficacy of systemically administered AONs, such as splice-switching AONs, depends on multiple factors such as administration route, biostability, biodistribution, intra-tissue distribution, uptake by target cells, and routing to the desired intracellular location (nucleus). Thus, there is a need for AONs with improved characteristics for treating, preventing and/or delaying DMD.

### SUMMARY

In one embodiment, provided herein is a hydroxyalkoxylated AON. In another embodiment, provided is a hydroxyalkoxylated AON consisting of one antisense oligonucleotide (AON) and one or two hydroxyalkoxy groups. In certain embodiments, the hydroxyalkoxy group comprises or consists of an ethylene glycol monomer, ethylene glycol oligomer or ethylene glycol polymer. In other embodiments, the AONs for use in the hydroxyalkoxylated AONs provided herein are represented by a nucleotide sequence comprising or consisting of:

any one of SEQ ID NO: 9-404, or

a fragment of any one of SEQ ID NO: 9-404, or

any one of SEQ ID NO: 9-404 with 1, 2, 3, 4, or 5 additional nucleotides, or

any one of SEQ ID NO: 9-404 with 1, 2, 3, 4, or 5 nucleotides missing from said SEQ ID NO, or

a nucleotide sequence which has at least 90%, 91%, 92%, 93%, 94%, 95%, 96%, 97%, 98% or 99%, or at least 95%, or at least 97%, identity with any one of SEQ ID NO: 9-404.

In certain embodiments, the hydroxyalkoxylated AON provided herein consists of 2′-O-methyl RNA monomers linked by phosphorothioate backbone linkages. In another embodiment, the hydroxyalkoxylated AON provided herein consists of 2′-O-methyl RNA monomers linked by phosphate backbone linkages. In another embodiment, the hydroxyalkoxylated AON provided herein consists of 2′-O-methyl RNA monomers linked by a mixture of phosphorothioate and phosphate backbone linkages.

In some embodiments, the AONs for use in the hydroxyalkoxylated AONs provided herein are complementary or reverse complementary to a portion of an exon in human dystrophin pre-mRNA. In some embodiments, the AONs for use in the hydroxyalkoxylated AONs provided herein are complementary to a portion of an exon in human dystrophin pre-mRNA. In certain embodiments, the AONs are complementary to a portion of one of exons 51, 45, 53, 52 or 55 of hyman dystrophin pre-mRNA. In certain embodiments, the AONs are complementary to a portion of exon 51 of hyman dystrophin pre-mRNA.

In further embodiments, provided is a pharmaceutical composition comprising a hydroxyalkoxylated AON provided herein and a pharmaceutically acceptable carrier.

In another embodiment, provided is a method of preventing, treating, and/or delaying Duchenne Muscular Dystrophy (DMD), comprising administering to a subject a hydroxyalkoxylated AON provided herein or a pharmaceutical composition provided herein.

In another embodiment, provided is a method of inducing skipping of exon 51 of the dystrophin pre-mRNA by contacting the dystrophin pre-mRNA with a hydroxyalkoxylated AON provided herein.

In some embodiments, linking an AON described herein to one or two hydroxyalkoxy moieties leads to a hydroxyalkoxylated AON that shows improved characteristics for treatment of genetic disorders, such as DMD, as compared to the AON lacking a hydroxyalkoxy group.

### BRIEF DESCRIPTION OF THE FIGURES

FIG. 1 shows dystrophin expression and exon skipping levels in quadriceps and heart of hDMDΔ52/mdx mice treated with the AON with SEQ ID NO: 22333 (“Cmpd 1”, 18 mg/kg) or the PS-linked hydroxyalkoxylated AON with SEQ ID NO: 22345 (“Cmpd 2 PS”, 18.7 mg/kg) compared to mice treated with vehicle in a 13-week treatment (by weekly intravenous tail vein injections).

FIG. 2 shows dystrophin expression and exon skipping levels in quadriceps of hDMDΔ52/mdx mice treated with the PS-linked hydroxyalkoxylated AON with SEQ ID NO: 22345 (Cmpd 2 PS, 9.4, 18.7 or 37.5 mg/kg) at 14 or 28 days post-dosing QW, Q2W or Q4W.

FIG. 3A shows effects of the AON with SEQ ID NO: 22333 (Cmpd 1) or the PS-linked or PO-linked hydroxyalkoxylated AON with SEQ ID NO: 22345 (Cmpd 2 PS or Cmpd 2 PO, respectively) on liver function measured by alkaline phosphatase (“ALP”), alanine aminotransferase (“ALT”) and aspartate transaminase (“AST”) levels in healthy CD-1 mice.

FIG. 3B shows effects of the AON with SEQ ID NO: 22333 (Cmpd 1) or the hydroxyalkoxylated AON with SEQ ID NO: 22345 (Cmpd 2 PS) on liver function measured by alanine aminotransferase (“ALT”) and aspartate transaminase (“AST”) levels in hDMDΔ52/mdx mice. FIG. 3C shows effects of the hydroxyalkoxylated AON with SEQ ID NO. 22345 (Cmpd 2 PS) on kidney function in hDMDΔ52/mdx mice.

FIG. 4 shows effects of the AON with SEQ ID NO: 22333 (Cmpd 1) or the hydroxyalkoxylated AON with SEQ ID NO: 22345 (Cmpd 2 PO) on complement parameters Bb and C3a.

FIG. 5 shows dose proportional plasma pK (AON concentration) of the AON with SEQ ID NO: 22333 (Cmpd 1) or the hydroxyalkoxylated AON with SEQ ID NO: 22345 (Cmpd 2 PO) at day 1, 22 and 50 post-dosing.

FIG. 6 shows significant improvement in functional motor endpoints (distance from wild-type) for the hydroxyalkoxylated AON with SEQ ID NO: 22345 (Cmpd 2 PS) in hDMDΔ52/mdx mice compared to vehicle in wild-type mice and hDMDΔ52/mdx mice.

FIG. 7 shows correction of biomarkers (nNOS, C4 binding protein, Fibrinogen g3) after treatment with the AON with SEQ ID NO: 22333 (Cmpd 1) in hDMDΔ52/mdx mice compared to vehicle in wild-type mice and hDMDΔ52/mdx mice.

FIG. 8 shows effects on body weight of hDMDΔ52/mdx mice treated with the AON with SEQ ID NO: 22333 (Cmpd 1) compared to the vehicle-treated wild-type and hDMDΔ52/mdx mice.

### DETAILED DESCRIPTION

### Definitions

As used herein, when the word “oligonucleotide” is used it may be replaced by “antisense oligonucleotide” and vice versa as defined herein unless otherwise indicated.

As used herein, the term “complementary” encompasses both forward complementary and reverse complementary sequences, as will be apparent to a skilled person from the context. When an oligonucleotide is complementary, it is understood that it can also be reverse complementary. As such, when “an oligonucleotide is complementary to” a target sequence is used, this means that said oligonucleotide is reverse complementary to said target sequence as the sequence of the oligonucleotide is the reverse complement of the target sequence, unless otherwise stated. When “an antisense oligonucleotide is complementary to” a target sequence is used, this means that said antisense oligonucleotide is complementary to said target sequence as the sequence of the antisense oligonucleotide is the reverse of the target sequence, unless otherwise stated.

As used herein, “hydroxyalkoxylated AON consisting of one antisense oligonucleotide and one or two hydroxyalkoxy groups” means that the antisense oligonucleotide is attached to one or two hydroxyalkoxy groups (as described in the section entitled “Hydroxyalkoxy Groups”) and hence form one molecule. In one embodiment, the linkage between the antisense oligonucleotide and a hydroxyalkoxy group is covalent. The linkage may be, in some embodiments, a phosphate (i.e., —P(O)(OH)—) linkage (“PO”) or, in other embodiments, a thiophosphate (i.e., —P(S)(OH)—) linkage (“PS”).

As used herein, “an oligonucleotide consisting of 2′-O-methyl RNA monomers linked by or connected through phosphorothioate backbone linkages” may be replaced by “an oligonucleotide consisting of 2′-O-methyl phosphorothioate RNA”. In this disclosure, such terms are synonymous.

As used herein, the expression “a hydroxyalkoxy group present at the 5′ or 3′ terminal monomer of an AON” may be interchanged throughout the document with “a hydroxyalkoxy group linked to the 5′ or 3′ terminal monomer of an AON” as they have an identical meaning in this context.

As used herein, “5′ or 3′ terminal monomer” has the same meaning as and can be used interchangeably with “the monomer at the 5′ or 3′ terminus”.

As used herein, unless otherwise specified, “a fragment of a SEQ ID NO:” means a nucleotide sequence comprising or consisting of at least 8, 9, 10, 11, 12, 13, 14, 15, 16, 17 or 18 contiguous nucleotides from said SEQ ID NO, or at least 10 contiguous nucleotides, or at least 16 contiguous nucleotides. As such, “a fragment of a SEQ ID NO:” means a nucleotide sequence which comprises or consists of said SEQ ID NO, wherein no more than 10, 9, 8, 7, 6, 5, 4, 3, 2 or 1 contiguous nucleotides are missing, no more than 8 contiguous nucleotides, or no more than 5 contiguous nucleotides are missing. In another embodiment, “a fragment of a SEQ ID NO:” means a nucleotide sequence comprising or consisting of an amount of contiguous nucleotides from said SEQ ID NO and wherein said amount of contiguous nucleotides is at least 50%, 60%, 70%, 80%, 90%, 91%, 92%, 93%, 94%, 95; 96%, 97%, 98% or 99% of the length of said SEQ ID NO. As such, “a fragment of a SEQ ID NO:” means a nucleotide sequence which comprises or consists of said SEQ ID NO, wherein an amount of contiguous nucleotides are missing and wherein said amount is no more than 50%, 45%, 40%, 35%, 30%, 25%, 20%, 15%, 10%, 5%, 4%, 3%, 2% or 1%, in one embodiment, no more than 20%, or no more than 10%, of the length of said SEQ ID NO.

As used herein, the term “(reverse) complementarity” means a stretch of nucleic acids that can hybridize to another stretch of nucleic acids under physiological conditions. An antisense strand is generally said to be complementary to a matching sense strand. In this context, an antisense oligonucleotide is complementary to its target. Hybridization conditions are defined herein. It is thus not absolutely required that all the bases in the region of complementarity are capable of pairing with bases in the opposing strand. For instance, when designing an antisense oligonucleotide, one may want to incorporate for instance a residue that does not base pair with the base on the complementary strand. Mismatches may to some extent be allowed, if under the circumstances in the cell, the stretch of nucleotides is capable of hybridizing to the complementary part.

As used herein, unless mentioned otherwise, the term “binds to” can be replaced with “complementary to”, “hybridizes to”, “overlaps with” and/or “targets” when used in the context of an antisense oligonucleotide which is complementary to a part of a pre-mRNA as identified herein. In this disclosure, such terms are synonymous. As used herein, “hybridizes” is used under physiological conditions in a cell. In one embodiment, the cell is a muscle cell unless otherwise indicated.

As used herein, “carbohydrate cluster” means a compound having one or more carbohydrate residues attached to a scaffold or hydroxyalkoxy group, (see, e.g., Maier et al., “Synthesis of Antisense Oligonucleotides Linked to a Multivalent Carbohydrate Cluster for Cellular Targeting,” Bioconjugate Chem., 2003, (14): 18-29; Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, (47): 5798-5808).

As used herein, “carbohydrate” means a naturally occurring carbohydrate, a modified carbohydrate, or a carbohydrate derivative.

As used herein, “modified carbohydrate” means a naturally occurring carbohydrate having one or more chemical modifications.

As used herein, “carbohydrate derivative” means any compound which may be synthesized using a naturally occurring carbohydrate as a starting material or intermediate.

As used herein, a “dystrophin pre-mRNA” means a pre-mRNA of a dystrophin gene coding for a dystrophin protein. A mutated dystrophin pre-mRNA corresponds to a pre-mRNA of a DMD patient with a mutation when compared to a wild type dystrophin pre-mRNA of a non-affected person, resulting in reduced levels or the absence of functional dystrophin (DMD).

As used herein, a “patient” means a subject having DMD as defined herein or a subject susceptible to develop DMD due to his genetic background.

As used herein, a “functional dystrophin” is a wild type dystrophin corresponding to a protein having the amino acid sequence as identified in SEQ ID NO: 1. As defined herein, a semi-functional dystrophin is a BMD-like dystrophin corresponding to a protein having an actin binding domain in its N terminal part (first 240 amino acids at the N terminus), a cysteine-rich domain (amino acid 3361 till 3685) and a C terminal domain (last 325 amino acids at the C terminus) each of these domains being present in a wild type dystrophin as known to the skilled person. The amino acids indicated herein correspond to amino acids of the wild type dystrophin being represented by SEQ ID NO: 1. In other words, a functional or a semi-functional dystrophin is a dystrophin which exhibits at least to some extent an activity of a wild type dystrophin. “At least to some extent” means at least 10%, 20%, 30%, 40%, 50%, 60%, 70%, 80%, 90%, 95% or 100% of a corresponding activity of a wild type functional dystrophin. In one embodiment, an activity of a functional dystrophin is binding to actin and to the dystrophin-associated glycoprotein complex (DGC or DAPC) (Ehmsen J et al, J. Cell Sci. 2002, 115 (Pt14): 2801-2803).

The terms “thymine” and “5-methyluracil” may be interchanged throughout the document. As used herein the expression “oligonucleotide comprises a 5-methylpyrimidine” means that at least one of the cytosine nucleobases of said oligonucleotide has being modified by substitution of the hydrogen at the 5-position of the pyrimidine ring with a methyl group, i.e. a 5-substituted cytosine, and/or that at least one of the uracil nucleobases of said oligonucleotide has been modified by substitution of the proton at the 5-position of the pyrimidine ring with a methyl group (i.e. a 5-methyluracil). As used herein, the expression “the substitution of a hydrogen with a methyl group in position 5 of the pyrimidine ring” may be replaced by the expression “the substitution of a pyrimidine with a 5-methylpyrimidine,” with pyrimidine referring to only uracil, only cytosine, or both.

As used herein, reference to an element by the indefinite article “a” or “an” does not exclude the possibility that more than one of the element is present, unless the context clearly requires that there be one and only one of the elements. The indefinite article “a” or “an” thus usually means “at least one”.

Each embodiment as identified herein may be combined together unless otherwise indicated. All patent, patent application publication and literature references cited in the present specification are hereby incorporated by reference in their entirety and for all intents and purposes.

When a structural formula or chemical name is understood by the skilled person to have chiral centers, yet no chirality is indicated, for each chiral center individual reference is made to all three of either the racemic mixture, the pure R enantiomer, and the pure S enantiomer.

Whenever a parameter of a substance is discussed herein, it is assumed that unless otherwise specified, the parameter is determined, measured, or manifested under physiological conditions. Physiological conditions are known to a person skilled in the art, and comprise aqueous solvent systems, atmospheric pressure, pH-values between 6 and 8, a temperature ranging from room temperature to about 37° C. (from about 20° C. to about 40° C.), and a suitable concentration of buffer salts or other components. It is understood that charge is often associated with equilibrium. A moiety that is said to carry or bear a charge is a moiety that will be found in a state where it bears or carries such a charge more often than that it does not bear or carry such a charge. As such, an atom that is indicated in this disclosure to be charged could be non-charged under specific conditions, and a neutral moiety could be charged under specific conditions, as is understood by a person skilled in the art.

As used herein, “modulation” refers to a perturbation of amount or quality of a function or activity when compared to the function or activity prior to modulation. For example, modulation includes the change, either an increase (stimulation or induction) or a decrease (inhibition or reduction) in dystrophin mRNA or protein as defined earlier herein. As a further example, modulation of expression can include perturbing splice site selection of pre-mRNA processing, resulting in a change in the amount of a particular splice-variant present compared to conditions that were not perturbed.

Generally, a substitution replaces one chemical group, which might be hydrogen, by another chemical group. When considering the carbon skeleton of organic molecules, an RNA monomer is inherently 2′-substituted because it has a hydroxyl group at its 2′-position. A DNA monomer would therefore not be 2′-substituted, and an RNA monomer can be seen as a 2′-substituted DNA monomer. When an RNA monomer in turn is 2′-substituted, this substitution can have replaced either the 2′-OH or the 2′-H. When an RNA monomer is 2′-O-substituted, this substitution replaces the H of the 2′-OH moiety. As a non-limiting example, 2′-O-methyl RNA is a 2′-substituted monomer (—OMe substitutes —H) and a 2′-substituted RNA monomer (—OMe substitutes —OH) and a 2′-O-substituted RNA monomer (-Me substitutes —H), while 2′-F RNA is a 2′-substituted RNA monomer (—F substitutes —OH or —H) yet not a 2′-O-substituted RNA monomer (2′-0 is either no longer present, or is not substituted). 2′-F RNA where F is substituted for 2′-OH is a 2′-F-2′-deoxy RNA, which is also a 2′-F DNA.

In one embodiment, a decrease or increase of a parameter to be assessed means a change of at least 5% of the value corresponding to that parameter. In another embodiment, a decrease or increase of the value means a change of at least 10%, at least 20%, at least 30%, at least 40%, at least 50%, at least 70%, at least 90%, or 100%. In this latter case, it can be the case that there is no longer a detectable value associated with the parameter.

The use of a substance as a medicament as described herein can also be interpreted as the use of said substance in the manufacture of a medicament. Similarly, whenever a substance is used for treatment or as a medicament, it can also be used for the manufacture of a medicament for treatment.

As used herein, the word “about” or “approximately” when used in association with a numerical value (e.g. about 10) means that the value may be the given value (of 10) more or less 0.1% of the value.

As will be understood by a skilled person, throughout this application, the terms “BNA”, “BNA scaffold”, “BNA nucleotide”, “BNA nucleoside”, “BNA modification”, or “BNA scaffold modification” may be replaced by conformationally restricted scaffold modification, locked scaffold modification, locked nucleotide, locked nucleoside, locked monomer, or Tm enhancing scaffold modification, or high-affinity modification and the like, as appropriate.

As used herein, “sequence identity” means a relationship between two or more nucleic acid (polynucleotide, nucleic acid or nucleotide or oligonucleotide) sequences, as determined by comparing the sequences. In one embodiment, sequence identity is calculated based on the full length of two given SEQ ID NO or on part thereof. Part thereof means at least 50%, 60%, 70%, 80%, 90%, or 100% of both SEQ ID NO. As used herein, “identity” also means the degree of sequence relatedness between nucleic acid sequences, as the case may be, as determined by the match between strings of such sequences.

Methods to determine identity are designed to give the largest match between the sequences tested. Methods to determine identity and similarity are codified in publicly available computer programs. Computer program methods to determine identity and similarity between two sequences include, e.g., the GCG program package (Devereux, J., et al., Nucleic Acids Research 12 (1): 387 (1984)), BestFit, BLASTN, and FASTA (Altschul, S. F. et al., J. Mol. Biol. 215:403-410 (1990)). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990)). The well-known Smith Waterman algorithm may also be used to determine identity.

Parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis.

Hybridization conditions for a nucleic acid molecules may have low or medium or high stringency (southern blotting procedures). Low or medium or high stringency conditions means pre-hybridization and hybridization at 42° C. in 5×SSPE, 0.3% SDS, 200 pg/ml sheared and denatured salmon sperm DNA, and either 25% or 35% or 50% formamide for low or medium or high stringencies respectively. Subsequently, the hybridization reaction is washed three times for 30 minutes each using 2×SSC, 0.2% SDS and either 55° C. or 65° C., or 75° C. for low or medium or high stringencies respectively.

Hydroxyalkoxylated AONs

In one embodiment, provided is a hydroxyalkoxylated AON consisting or consisting essentially of one antisense oligonucleotide (AON) and an ethylene glycol monomer, ethylene glycol oligomer or ethylene glycol polymer, wherein the antisense oligonucleotide is represented by a nucleotide sequence comprising or consisting of:

any one of SEQ ID NO: 9-404, or any one of SEQ ID NO: 9-11, or

a fragment of any one of SEQ ID NO: 9-404, or of any one of SEQ ID NO: 9-11 or

any one of SEQ ID NO: 9-404, or any one of SEQ ID NO: 9-11, with 1, 2, 3, 4, or 5 additional nucleotides, or

any one of SEQ ID NO: 9-404, or any one of SEQ ID NO: 9-11, with 1, 2, 3, 4, or 5 nucleotides missing from said SEQ ID NO, or

a nucleotide sequence which has at least 90%, 91%, 92%, 93%, 94%, 95%, 96%, 97%, 98% or 99%, or at least 95%, or at least 97%, identity with any one of SEQ ID NO: 9-404, or with any one of SEQ ID NO: 9-11,

and wherein said AON consists of 2′-O-methyl RNA monomers linked by phosphorothioate backbone linkages, for use as a medicament, or for treating, preventing and/or delaying Duchenne Muscular Dystrophy (DMD), or for inducing skipping of exon 51 of the dystrophin pre-mRNA. In one embodiment, said exon 51 of dystrophin pre-mRNA is from a human and is represented by a nucleotide sequence with SEQ ID NO: 2.

In one embodiment, said 1, 2, 3, 4 or 5 additional nucleotides may be present at the 5′ and/or 3′ terminus of any one of SEQ ID NO: 9-404. In another embodiment, said 1, 2, 3, 4 or 5 missing nucleotides may be nucleotides present at the 5′ and/or 3′ terminus of any one of SEQ ID NO: 9-404.

In another embodiment, provided is a hydroxyalkoxylated AON consisting or consisting essentially of one antisense oligonucleotide (AON) and one or two hydroxyalkoxy groups, said hydroxyalkoxy groups comprise or consist of an ethylene glycol monomer, ethylene glycol oligomer or ethylene glycol polymer, wherein said antisense oligonucleotide is represented by a nucleotide sequence comprising or consisting of:

any one of SEQ ID NO: 9-404, or any one of SEQ ID NO: 9-11, or

a fragment of any one of SEQ ID NO: 9-404, or any one of SEQ ID NO: 9-11 or

any one of SEQ ID NO: 9-404, or any one of SEQ ID NO: 9-11, with 1, 2, 3, 4, or 5 additional nucleotides, or

any one of SEQ ID NO: 9-404, or any one with SEQ ID NO: 9-11, with 1, 2, 3, 4, or 5 nucleotides missing from said SEQ ID NO, or

a nucleotide sequence which has at least 90%, 91%, 92%, 93%, 94%, 95%, 96%, 97%, 98% or 99%, or at least 95%, or at least 97%, identity with any one of SEQ ID NO: 9-404, or with any one of SEQ ID NO: 9-11,

and wherein said AON consists of 2′-O-methyl RNA monomers linked by phosphorothioate backbone linkages, for use as a medicament, or for treating, preventing and/or delaying Duchenne Muscular Dystrophy (DMD), or for inducing skipping of exon 51 of the dystrophin pre-mRNA. In one embodiment, said exon 51 of dystrophin pre-mRNA is from a human and is represented by a nucleotide sequence with SEQ ID NO: 2.

In one embodiment, said 1, 2, 3, 4 or 5 additional nucleotides may be present at the 5′ and/or 3′ terminus of any one of SEQ ID NO: 9-404. In another embodiment, said 1, 2, 3, 4 or 5 missing nucleotides may be nucleotides present at the 5′ and/or 3′ terminus of any one of SEQ ID NO: 9-404.

In some embodiments, a hydroxyalkoxy group of the hydroxyalkoxylated AON provided herein is present at the 5′ terminal monomer or the 3′ terminal monomer of said antisense oligonucleotide, or a first hydroxyalkoxy group is present at the 5′ terminal monomer and a second hydroxyalkoxy group is present at the 3′ terminal monomer of said antisense oligonucleotide. In some embodiments, a hydroxyalkoxy group of the hydroxyalkoxylated AON as described herein is present at the 5′ terminal monomer of said antisense oligonucleotide. In some embodiments, at least one hydroxyalkoxy group, or all hydroxyalkoxy groups of the hydroxyalkoxylated AON, comprises or consists of an ethylene glycol monomer, ethylene glycol oligomer or ethylene glycol polymer (as described in the section “Hydroxyalkoxy Groups”).

In one embodiment, provided is a hydroxyalkoxylated AON consisting or consisting essentially of one antisense oligonucleotide (AON) and one hydroxyalkoxy group (as described in the section “Hydroxyalkoxy Groups”), said hydroxyalkoxy group comprises or consists of an ethylene glycol monomer, ethylene glycol oligomer or ethylene glycol polymer, wherein said AON is represented by a nucleotide sequence as defined herein and wherein said AON consists of 2′-O-methyl RNA monomers linked by phosphorothioate backbone linkages, for use as a medicament, or for treating, preventing and/or delaying Duchenne Muscular Dystrophy (DMD), or for inducing skipping of exon 51 of the dystrophin pre-mRNA. In one embodiment, said exon 51 of dystrophin pre-mRNA is from a human and is represented by a nucleotide sequence with SEQ ID NO: 2. In one embodiment, said hydroxyalkoxy group is linked to the 5′ terminal monomer or the 3′ terminal monomer of said AON. In one embodiment, said hydroxyalkoxy group is linked to the 5′ terminal monomer of said AON. In another embodiment, said hydroxyalkoxy group is linked to the 3′ terminal monomer of said AON. In one embodiment, the linkage between the antisense oligonucleotide and a hydroxyalkoxy group is covalent.

In one embodiment, provided is a hydroxyalkoxylated AON consisting or consisting essentially of one antisense oligonucleotide (AON) and two hydroxyalkoxy groups (as described in the section “Hydroxyalkoxy Groups”), wherein at least one hydroxyalkoxy group, or both hydroxyalkoxy groups, comprises or consists of an ethylene glycol monomer, ethylene glycol oligomer or ethylene glycol polymer, wherein said AON is represented by a nucleotide sequence as defined herein and wherein said AON consists of 2′-O-methyl RNA monomers linked by phosphorothioate backbone linkages, for use as a medicament, or for treating, preventing and/or delaying Duchenne Muscular Dystrophy (DMD). In one embodiment, the hydroxyalkoxylated AON induces skipping of exon 51 of the dystrophin pre-mRNA. In one embodiment, said exon 51 of dystrophin pre-mRNA is from a human and is represented by a nucleotide sequence with SEQ ID NO: 2. In one embodiment, the first hydroxyalkoxy group is linked to the 5′ terminal monomer and the second hydroxyalkoxy group is linked to the 3′ terminal monomer of said AON. In one embodiment, the linkage between the antisense oligonucleotide and a hydroxyalkoxy group is covalent.

Dystrophin Exon

Throughout this application, unless explicitly specified otherwise, a hydroxyalkoxylated AON is for skipping exon 51 of dystrophin pre-mRNA.

In one embodiment, said exon 51 of dystrophin pre-mRNA is from a human and is represented by the following nucleotide sequence:

(SEQ ID NO: 2)
5′-CUCCUACUCAGACUGUUACUCUGGUGACACAACCUGUGGUUACUAAG
GAAACUGCCAUCUCCAAACUAGAAAUGCCAUCUUCCUUGAUGUUGGAGGU
ACCUGCUCUGGCAGAUUUCAACCGGGCUUGGACAGAACUUACCGACUGGC
UUUCUCUGCUUGAUCAAGUUAUAAAAUCACAGAGGGUGAUGGUGGGUGAC
CUUGAGGAUAUCAACGAGAUGAUCAUCAAGCAGAAG-3′.

Antisense Oligonucleotide of the Hydroxyalkoxylated AON

In one embodiment, provided is a hydroxyalkoxylated AON consisting or consisting essentially of one antisense oligonucleotide and one or two hydroxyalkoxy groups (as described in the section entitled “Hydroxyalkoxy Groups”), wherein said antisense oligonucleotide is represented by a nucleotide sequence comprising or consisting of any one of SEQ ID NO: 9-404, or any one of SEQ ID NO: 9-11, or a fragment thereof, and wherein said antisense oligonucleotide consists of 2′-O-methyl RNA monomers linked by phosphorothioate backbone linkages. The antisense oligonucleotide of the hydroxyalkoxylated AON may additionally have any of the chemistries disclosed herein or combinations thereof (as described in the section “Chemical modifications of the antisense oligonucleotide of the hydroxyalkoxylated AON”).

In one embodiment, said fragment of a SEQ ID NO has dystrophin pre-mRNA exon 51 skipping activity.

In another embodiment provided is a hydroxyalkoxylated AON consisting or consisting essentially of one antisense oligonucleotide and one or two hydroxyalkoxy groups (as described in the section entitled “Hydroxyalkoxy Groups”), wherein said antisense oligonucleotide is represented by a nucleotide sequence comprising or consisting of a nucleotide sequence which has at least 90%, 91%, 92%, 93%, 94%, 95%, 96%, 97%, 98% or 99%, in one embodiment at least 95%, or at least 97%, identity with any one of SEQ ID NO: 9-404, or any one of SEQ ID NO: 9-11 and wherein said antisense oligonucleotide consists of 2′-O-methyl RNA monomers linked by phosphorothioate backbone linkages. In one embodiment, said antisense oligonucleotide has dystrophin pre-mRNA exon 51 skipping activity.

The antisense oligonucleotide of the hydroxyalkoxylated AON may additionally have any of the modifications disclosed herein or combinations thereof (as described in the section “Chemical modifications of the antisense oligonucleotide of the hydroxyalkoxylated AON”).

In one embodiment, provided is a hydroxyalkoxylated AON, for skipping exon 51, consisting or consisting essentially of one antisense oligonucleotide (AON) and one or two hydroxyalkoxy groups (as described in the section entitled “Hydroxyalkoxy Groups”). In one embodiment, said hydroxyalkoxy group is a triethylene glycol (TEG) group, wherein said antisense oligonucleotide is represented by a nucleotide sequence comprising or consisting of:

any one of SEQ ID NO: 9-404, or any one of SEQ ID NO: 9-11, or

a fragment of any one of SEQ ID NO: 9-404, or any one of SEQ ID NO: 9-11 or

any one of SEQ ID NO: 9-404, or any one of SEQ ID NO: 9-11, with 1, 2, 3, 4, or 5 additional nucleotides, or

any one of SEQ ID NO: 9-404, or any one of SEQ ID NO: 9-11, with 1, 2, 3, 4, or 5 nucleotides missing from said SEQ ID NO, or

a nucleotide sequence which has at least 90%, 91%, 92%, 93%, 94%, 95%, 96%, 97%, 98% or 99%, or at least 95%, or at least 97%, identity with any on of SEQ ID NO: 9-404, or any one of SEQ ID NO: 9-11,

and wherein said AON consists of 2′-O-methyl RNA monomers linked by phosphorothioate backbone linkages.

In one embodiment, said “1, 2, 3, 4 or 5 additional nucleotides” may be present at the 5′ and/or 3′ terminus of any one of SEQ ID NO: 9-404.

In another embodiment, said “1, 2, 3, 4 or 5 missing nucleotides” may be nucleotides missing at the 5′ and/or 3′ terminus of any one of SEQ ID NO: 9-404.

The antisense oligonucleotide of the hydroxyalkoxylated AON may additionally have any of the chemistries disclosed herein or combinations thereof (as described in the section “Chemical modifications of the antisense oligonucleotide of the hydroxyalkoxylated AON”).

In some embodiments, there are 1 or 2 mismatch(es) in an oligonucleotide of 20 nucleotides or 1 to 4 mismatches in an oligonucleotide of 40 nucleotides as defined herein. In one embodiment, in an oligonucleotide of 10 to 33 nucleotides, 0, 1, 2 or 3 mismatches are present. In another embodiment, 0, 1 or 2 mismatches are present. In further embodiments, in an oligonucleotide of 16 to 22 nucleotides, 0, 1 or 2 mismatches are present, or 0 or 1 mismatch(es) is(are) present.

In another embodiment, a hydroxyalkoxylated AON as described herein is for skipping exon 51 of the pre-mRNA of dystrophin.

In one embodiment, a hydroxyalkoxylated AON as described herein is for skipping exon 51 of the pre-mRNA of dystrophin, and consists or consists essentially of one antisense oligonucleotide and a hydroxyalkoxy group (as described in the section entitled “Hydroxyalkoxy Groups”), said hydroxyalkoxy group is triethylene glycol (TEG), wherein said antisense oligonucleotide is represented by a nucleotide sequence comprising or consisting of any one of SEQ ID NO: 9-404, and wherein said antisense oligonucleotide consists of 2′-O-methyl RNA monomers linked by phosphorothioate backbone linkages.

In certain embodiments, a hydroxyalkoxy group (as described in the section entitled “Hydroxyalkoxy Groups”) of the hydroxyalkoxylated AON as described herein may be present at the 5′ terminal monomer or at the 3′ terminal monomer of the antisense oligonucleotide. If the hydroxyalkoxylated AON consists of one antisense oligonucleotide and two hydroxyalkoxy groups, a first hydroxyalkoxy group is present at the 5′ terminal monomer and a second hydroxyalkoxy group is present at the 3′ terminal monomer of said antisense oligonucleotide.

In one embodiment, a hydroxyalkoxylated AON consisting or consisting essentially of one antisense oligonucleotide and one or two hydroxyalkoxy groups (as described in the section “Hydroxyalkoxy Groups”), is represented by:

any one of SEQ ID NO: 405-800, if a hydroxyalkoxy group is present at the 5′ terminal monomer of the antisense oligonucleotide represented by any one of SEQ ID NO: 9-404, respectively,

or any one of SEQ ID NO: 405-407, if a hydroxyalkoxy group is present at the 5′ terminal monomer of the antisense oligonucleotide represented by any one of SEQ ID NO: 9-11, respectively; or

any one of SEQ ID NO: 801-1196, if a hydroxyalkoxy group is present at the 3′ terminal monomer of the antisense oligonucleotide represented by any one of SEQ ID NO: 9-404, respectively,

or any one of SEQ ID NO: 801-803, if a hydroxyalkoxy group is present at the 3′ terminal monomer of the antisense oligonucleotide represented by any one of SEQ ID NO: 9-11, respectively; or

any one of SEQ ID NO: 1197-1592, if a first hydroxyalkoxy group is present at the 5′ terminal monomer and a second hydroxyalkoxy group is present at the 3′ terminal monomer of the antisense oligonucleotide represented by any one of SEQ ID NO: 9-404,

or any one of SEQ ID NO: 1197-1199, if a first hydroxyalkoxy group is present at the 5′ terminal monomer and a second hydroxyalkoxy group is present at the 3′ terminal monomer of the antisense oligonucleotide represented by any one of SEQ ID NO: 9-11, respectively;

wherein said hydroxyalkoxy group, represented by n in the sequence listing, is a triethylene glycol (TEG) group.

In one embodiment, a hydroxyalkoxylated AON as described herein consists or consists essentially of one antisense oligonucleotide, represented by any one of SEQ ID NO: 9-11, and one or two hydroxyalkoxy groups (as described in the section “Hydroxyalkoxy Groups”), and can be represented by:

SEQ ID NO: 405
(nUCAAGGAAGAUGGCAUUUCU),
SEQ ID NO: 406
(nUCAAGGAAGAUGGCAUUUCUAG),
SEQ ID NO: 407
(nGGUAAGUUCUGUCCAAGC),

if a hydroxyalkoxy group is present at the 5′ terminal monomer of the antisense oligonucleotide represented by any one of SEQ ID NO: 9-11, respectively; or

SEQ ID NO: 801 (UCAAGGAAGAUGGCAUUUCUn), SEQ ID NO: 802 (UCAAGGAAGAUGGCAUUUCUAGn), SEQ ID NO: 803 (GGUAAGUUCUGUCCAAGCn), if a hydroxyalkoxy group is present at the 3′ terminal monomer of the antisense oligonucleotide represented by any one of SEQ ID NO: 9-11, respectively; or

SEQ ID NO: 1197 (nUCAAGGAAGAUGGCAUUUCUn), SEQ ID NO: 1198 (nUCAAGGAAGAUGGCAUUUCUAGn) and SEQ ID NO: 1199 (nGGUAAGUUCUGUCCAAGCn), if a first hydroxyalkoxy group is present at the 5′ terminal monomer and a second hydroxyalkoxy group is present at the 3′ terminal monomer of the antisense oligonucleotide represented by any one of SEQ ID NO: 9-11, respectively;

wherein said hydroxyalkoxy group, represented by n, is a triethylene glycol (TEG) group.

In one embodiment, said antisense oligonucleotide of the hydroxyalkoxylated AON described herein has a length of 8 to 33 nucleotides, of 12 to 24 nucleotides, of 13 to 23 nucleotides, or of 16 to 22 nucleotides. However, the length of said antisense oligonucleotide may be at least 8, 9, 10, 11, 12, 13, 14, 15, 16, 17, 18, 19, 20, 21, 22, 23, 24, 25, 26, 27, 28, 29, 30, 31 or 32 nucleotides.

In one embodiment, the antisense oligonucleotide of the hydroxyalkoxylated AON described herein is represented by a nucleotide sequence comprising any one of SEQ ID NO: 9-407, or any one of SEQ ID NO: 9-11, or a fragment thereof, wherein said nucleotide sequence or fragment is complementary to or binds to or targets or hybridizes to or overlaps with at least a part of an exonic splicing enhancer (ESE) sequence located within exon 51 of dystrophin pre-mRNA, wherein said exonic splicing enhancer (ESE) sequence is represented by SEQ ID NO: 3 (GGACAGAACUU) or SEQ ID NO: 5 (AUCUUC). Said binding or targeted or hybridized part may be at least 50% of the length of the antisense oligonucleotide, or at least 60%, or at least 70%, or at least 80%, or at least 90%, or at least 95%, or at least 98% and up to 100%. In one embodiment, said exonic splicing enhancer (ESE) sequence is represented by SEQ ID NO: 3. In another embodiment, said exonic splicing enhancer (ESE) sequence is represented by SEQ ID NO: 5.

In another embodiment, a hydroxyalkoxylated AON is provided wherein the antisense oligonucleotide is represented by a nucleotide sequence as defined herein and which has at least 95% identity with a contiguous stretch of at least 4, 5, 6, 7, 8, 9, 10 or 11 nucleotides, at least 8 nucleotides, at least 10 nucleotides, or all nucleotides of a reverse-complementary ESE sequence, wherein said reverse-complementary ESE sequence is SEQ ID NO: 4 or SEQ ID NO: 6. Said SEQ ID NO: 4 and SEQ ID NO: 6 represent the reverse-complement sequence of SEQ ID NO: 3 and SEQ ID NO: 5, respectively. Said contiguous stretch may be interrupted by one, two, three, four or more gaps as long as the identity percentage over the whole region is at least 95%, at least 96%, 97%, 98%, 99% or 100%. In one embodiment, the identity percentage over the whole region is at least 97%.

In one embodiment, said reverse-complementary ESE sequence is SEQ ID NO: 4. Examples of antisense oligonucleotides for use in the hydroxyalkoxylated AONs provided herein are represented by SEQ ID NO: 11-194.

In one embodiment, a hydroxyalkoxylated AON is provided consisting or consisting essentially of one antisense oligonucleotide and one or two hydroxyalkoxy groups, said hydroxyalkoxy groups comprise or consist of an ethylene glycol monomer, ethylene glycol oligomer or ethylene glycol polymer, wherein said antisense oligonucleotide is represented by a nucleotide sequence comprising or consisting of:

any one of SEQ ID NO: 11-194, or SEQ ID NO: 11, or

a fragment of any one of SEQ ID NO: 11-194, or SEQ ID NO: 11 or

any one of SEQ ID NO: 11-194, or SEQ ID NO: 11, with 1, 2, 3, 4, or 5 additional nucleotides, or

any one of SEQ ID NO: 11-194, or SEQ ID NO: 11, with 1, 2, 3, 4, or 5 nucleotides missing from said SEQ ID NO, or

a nucleotide sequence which has at least 90%, 91%, 92%, 93%, 94%, 95%, 96%, 97%, 98% or 99%, or at least 95%, or at least 97%, identity with any one of SEQ ID NO: 11-194, with SEQ ID NO: 11,

and wherein said AON consists of 2′-O-methyl RNA monomers linked by phosphorothioate backbone linkages, for use as a medicament, or for treating, preventing and/or delaying Duchenne Muscular Dystrophy (DMD), or for inducing skipping of exon 51 of the dystrophin pre-mRNA. In one embodiment, said exon 51 of dystrophin pre-mRNA is from a human and is represented by a nucleotide sequence with SEQ ID NO: 2.

In one embodiment, said 1, 2, 3, 4 or 5 additional nucleotides are present at the 5′ and/or 3′ terminus of any one of SEQ ID NO: 11-194. In another embodiment, said 1, 2, 3, 4 or 5 missing nucleotides are nucleotides present at the 5′ and/or 3′ terminus of any one of SEQ ID NO: 11-194.

In another embodiment, said reverse-complementary ESE sequences is SEQ ID NO: 6. Examples of antisense oligonucleotides for use in the hydroxyalkoxylated AONs provided herein are represented by SEQ ID NO: 195-395. It should be noted that SEQ ID NO: 195-196 are identical to SEQ ID NO: 9-10, respectively, and are interchangeable.

In one embodiment, provided is a hydroxyalkoxylated AON consisting or consisting essentially of one antisense oligonucleotide and one or two hydroxyalkoxy groups, said hydroxyalkoxy groups comprise or consist of an ethylene glycol monomer, ethylene glycol oligomer or ethylene glycol polymer, wherein said antisense oligonucleotide is represented by a nucleotide sequence comprising or consisting of:

any one of SEQ ID NO: 195-395, or any one of SEQ ID NO: 195-196, or

a fragment of any one of SEQ ID NO: 195-395, or any one of SEQ ID NO: 195-196 or

any one of SEQ ID NO: 195-395, or any one of SEQ ID NO: 195-196, with 1, 2, 3, 4, or 5 additional nucleotides, or

any one of SEQ ID NO: 195-395, or any one with SEQ ID NO: 195-196, with 1, 2, 3, 4, or 5 nucleotides missing from said SEQ ID NO, or

a nucleotide sequence which has at least 90%, 91%, 92%, 93%, 94%, 95%, 96%, 97%, 98% or 99%, or at least 95%, or at least 97%, identity with any one of SEQ ID NO: 195-395, or with any one of SEQ ID NO: 195-196,

and wherein said AON consists of 2′-O-methyl RNA monomers linked by phosphorothioate backbone linkages, for use as a medicament, or for treating, preventing and/or delaying Duchenne Muscular Dystrophy (DMD), or for inducing skipping of exon 51 of the dystrophin pre-mRNA. In one embodiment, said exon 51 of dystrophin pre-mRNA is from a human and is represented by a nucleotide sequence with SEQ ID NO: 2.

In one embodiment, said 1, 2, 3, 4 or 5 additional nucleotides are present at the 5′ and/or 3′ terminus of any one of SEQ ID NO: 195-395. In another embodiment, said 1, 2, 3, 4 or 5 missing nucleotides may be nucleotides present at the 5′ and/or 3′ terminus of any one of SEQ ID NO: 195-395.

Chemical Modifications of the Antisense Oligonucleotide of the Hydroxyalkoxylated AON

In one embodiment, the antisense oligonucleotide of the hydroxyalkoxylated AON provided herein consists of 2′-O-methyl RNA monomers linked by phosphorothioate backbone linkages and said antisense oligonucleotide is represented by a nucleotide sequence as defined herein (as described in the sections entitled “Hydroxyalkoxylated AON” and “Antisense oligonucleotide of the hydroxyalkoxylated AON”). In another embodiment, the phosphorothioate backbone linkages of said AON are chirally pure as described in, e.g., WO 2014/010250 (WaVe Life Sciences).

In another embodiment, the antisense oligonucleotide of the hydroxyalkoxylated AON can further comprise or consist of any chemical modification, or any combination thereof, as described herein. In one embodiment, a hydroxyalkoxylated AON described herein wherein said antisense oligonucleotide comprises or consists of any chemical modification, or any combination thereof, as described herein has an exon skipping activity that is at least as effective as said compound without said chemical modification or combination thereof. In one embodiment, said exon skipping activity is higher than said hydroxyalkoxylated AON without said chemical modification or combination thereof.

In one embodiment, said antisense oligonucleotide of the hydroxyalkoxylated AON is single stranded. The skilled person will understand that a single stranded oligonucleotide may form an internal double stranded structure. However, this oligonucleotide is still regarded as a single stranded oligonucleotide in this context.

In one embodiment, provided is a hydroxyalkoxylated AON wherein the antisense oligonucleotide consists of 2′-O-methyl RNA monomers linked by phosphorothioate backbone linkages and further comprise or consists of:

a 5-methylcytosine and/or a 5-methyluracil base, and/or

at least one monomer comprising a bicyclic nucleic acid (BNA) scaffold modification.

As known to the skilled person, an oligonucleotide such as an RNA oligonucleotide generally consists of repeating monomers. Such a monomer is most often a nucleotide or a nucleotide analogue. The most common naturally occurring nucleotides in RNA are adenosine monophosphate, cytidine monophosphate, guanosine monophosphate, thymidine monophosphate, and uridine monophosphate. These consist of a pentose sugar ribose, a 5′-linked phosphate group which is linked via a phosphate ester, and a 1′-linked base. The sugar connects the base and the phosphate, and is therefore often referred to as the scaffold of the nucleotide. A modification in the pentose sugar is therefore often referred to as a scaffold modification. For severe modifications, the original pentose sugar might be replaced in its entirety by another moiety that similarly connects the base and the phosphate. It is therefore understood that while a pentose sugar is often a scaffold, a scaffold is not necessarily a pentose sugar.

A base, sometimes called a nucleobase, is generally adenine, cytosine, guanine, thymine, or uracil, or a derivative thereof. Cytosine, thymine, and uracil are pyrimidine bases, and are generally linked to the scaffold through their 3′-nitrogen. Adenine and guanine are purine bases, and are generally linked to the scaffold through their 9′-nitrogen.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted to this copolymer, namely to the scaffold moieties. Because of this characteristic, the alternating copolymer formed by linked monomers of an oligonucleotide is often called the backbone of the oligonucleotide. Because the phosphodiester bonds connect neighboring monomers together, they are often referred to as backbone linkages. It is understood that when a phosphate group is modified so that it is instead an analogous moiety such as a phosphorothioate, such a moiety is still referred to as the backbone linkage of the monomer. This is referred to as a backbone linkage modification. In general terms, the backbone of an oligonucleotide is thus comprised of alternating scaffolds and backbone linkages.

In one embodiment, the antisense oligonucleotide of the hydroxyalkoxylated AON provided herein comprises a base modification that increases binding affinity to target strands, increases melting temperature of the resulting duplex of said oligonucleotide with its target, and/or decreases immunostimulatory effects, and/or increases biostability, and/or improves biodistribution and/or intra-tissue distribution, and/or cellular uptake and trafficking.

In one embodiment, said antisense oligonucleotide of the hydroxyalkoxylated AON provided herein comprises a 5-methylpyrimidine. In certain embodiments, the 5-methylpyrimidine is selected from 5-methylcytosine and/or 5-methyluracil and/or thymine, in which thymine is identical to 5-methyluracil. In other embodiments, if said oligonucleotide comprises 1, 2, 3, 4, 5, 6, 7, 8, 9, or more cytosines and/or uracils, at least 1, 2, 3, 4, 5, 6, 7, 8, 9, or more cytosines and/or uracils respectively have been modified this way.

In one embodiment, said antisense oligonucleotide of the hydroxyalkoxylated AON provided herein comprises at least one of either a 5-methylcytosine base or a 5-methyluracil base. In one embodiment, said antisense oligonucleotide of said hydroxyalkoxylated AON is provided wherein all cytosine bases are 5-methylcytosine bases, and optionally at least one uracil base is a 5-methyluracil base. In another embodiment, said antisense oligonucleotide of said hydroxyalkoxylated AON is provided wherein all cytosine bases are 5-methylcytosine bases, and optionally wherein also all uracil bases of said AON are 5-methyluracil bases.

In one embodiment, antisense oligonucleotides of the hydroxyalkoxylated AON described herein can be represented by any one of SEQ ID NO: 1593-1988, which correspond to any one of unmodified sequences SEQ ID NO: 9-404, respectively, wherein all cytosine bases are 5-methylcytosine bases.

In one embodiment, antisense oligonucleotides of the hydroxyalkoxylated AON described herein can be represented by any one of SEQ ID NO: 1593-1595, which correspond to any one of unmodified sequences SEQ ID NO: 9-11, respectively, wherein all cytosine bases are 5-methylcytosine bases.

In one embodiment, provided is a hydroxyalkoxylated AON, for skipping exon 51 of the pre-mRNA of dystrophin, consisting or consisting essentially of one antisense oligonucleotide and one or two hydroxyalkoxy groups (as described in the section “Hydroxyalkoxy Groups”), wherein said antisense oligonucleotide is represented by a nucleotide sequence comprising or consisting of any one of SEQ ID NO: 1593-1988 (derived from any one of SEQ ID NO: 9-404, wherein all cytosine bases are 5-methylcytosine bases), or any one of SEQ ID NO: 1593-1595 (derived from any one of SEQ ID NO: 9-11, wherein all cytosine bases are 5-methylcytosine bases), and wherein said hydroxyalkoxylated AON can be represented by:

any one of SEQ ID NO: 1989-2384, or any one of SEQ ID NO: 1989-1991, if a hydroxyalkoxy group is present at the 5′ terminal monomer of the antisense oligonucleotide; or

any one of SEQ ID NO: 2385-2780, or any one of SEQ ID NO: 2385-2387, if a hydroxyalkoxy group is present at the 3′ terminal monomer of the antisense oligonucleotide; or

any one of SEQ ID NO: 2781-3176, or any one of SEQ ID NO: 2781-2783, if a first hydroxyalkoxy group is present at the 5′ terminal monomer and a second hydroxyalkoxy group is present at the 3′ terminal monomer of the antisense oligonucleotide;

wherein said hydroxyalkoxy group, represented by n in the sequence listing, is a triethylene glycol (TEG) group.

In one embodiment, hydroxyalkoxylated AONs provided herein consist or consist essentially of one antisense oligonucleotide and one or two hydroxyalkoxy groups (as described in the section “Hydroxyalkoxy Groups”), wherein said antisense oligonucleotide is represented by a nucleotide sequence comprising or consisting of any one of SEQ ID NO: 1593-1595 (derived from any one of SEQ ID NO: 9-11, wherein all cytosine bases are 5-methylcytosine bases) and wherein said hydroxyalkoxylated AON can be represented by:

SEQ ID NO: 1989 (nUC*AAGGAAGAUGGC*AUUUC*U), SEQ ID NO: 1990 (nUC*AAGGAAGAUGGC*AUUUC*UAG), or SEQ ID NO: 1991 (nGGUAAGUUC*UGUC*C*AAGC*), if a hydroxyalkoxy group is present at the 5′ terminal monomer of the antisense oligonucleotide; or

SEQ ID NO: 2385 (UC*AAGGAAGAUGGC*AUUUC*Un), SEQ ID NO: 2386 (UC*AAGGAAGAUGGC*AUUUC*UAGn), or SEQ ID NO: 2387 (GGUAAGUUC*UGUC*C*AAGC*n), if a hydroxyalkoxy group is present at the 3′ terminal monomer of the antisense oligonucleotide; or

SEQ ID NO: 2781 (nUC*AAGGAAGAUGGC*AUUUC*Un), SEQ ID NO: 2782 (nUC*AAGGAAGAUGGC*AUUUC*UAGn) or SEQ ID NO: 2783 (nGGUAAGUUC*UGUC*C*AAGC*n), if a first hydroxyalkoxy group is present at the 5′ terminal monomer and a second hydroxyalkoxy group is present at the 3′ terminal monomer of the antisense oligonucleotide;

wherein said hydroxyalkoxy group, represented by n, is a triethylene glycol (TEG) group, and wherein C* is 5-methylcytosine.

In one embodiment, the antisense oligonucleotide of the hydroxyalkoxylated AON provided herein comprises a scaffold modification that increases binding affinity to target strands, and/or increases melting temperature of the resulting duplex of said first and/or second oligonucleotide with its target, and/or decreases immunostimulatory effects, and/or increases biostability, and/or improves biodistribution and/or intra-tissue distribution, and/or improves cellular uptake and trafficking.

In one embodiment, provided are those scaffold modifications that result in a bicyclic nucleic acid (BNA) monomer. A bicyclic scaffold is, in one embodiment, a pentose-derived scaffold that has been chemically altered to conformationally restrict the scaffold, leading to the improved effects above. Non-limiting examples of bicyclic scaffolds are scaffolds where a first cycle such as a pentose ring forms a spirane with a further cyclic moiety so that both cycles share only one atom, scaffolds where a first cycle such as a pentose cycle is fused to a further cyclic moiety so that both cycles share two adjacent atoms, and scaffolds where a first cycle such as a pentose cycle forms a bridged compound through a moiety that is linked to the first cyclic moiety at two non-adjacent atoms. Such non-adjacent atoms are referred to as bridgehead atoms. Bridged compounds comprise multiple cycles, each of which overlap over at least three atoms. A compound with two cycles wherein those cycles overlap over only two atoms is a fused compound instead. In some bridged compounds, the smallest link between two bridgehead atoms is referred to as the bridging moiety, or as the bridge moiety. In other bridged compounds, when one cycle is a characteristic cycle such as the pentose cycle of a nucleotide, the moiety that is not constitutive to that characteristic cycle is referred to as the bridging moiety. It follows that the nomenclature of bridged bicyclic compounds is context-dependent.

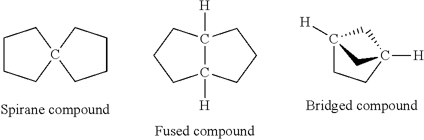 [SMILES: C1CCC2(C1)CCCC2 ; C1CC2CCCC2C1 ; C1C[C@H]2C[C@@H]1C2]

Bicyclic compounds can comprise additional cycles. A bicyclic compound contains at least two cycles, and said two cycles constitute a spirane, a fused system, or a bridged system, or a combination thereof. In one embodiment, not encompassed are scaffold modifications where two independent cycles are linked via a non-cyclic group, so as to not form a spirane, fused compound, or bridged compound. In one embodiment, bicyclic compounds are fused and bridged compounds. In some embodiments, a bicyclic nucleic acid monomer (BNA) is a bridged nucleic acid monomer.

In one embodiment, provided is a hydroxyalkoxylated AON wherein each occurrence of said bicyclic nucleic acid (BNA) scaffold modification in said antisense oligonucleotide results in a monomer that is independently chosen from the group consisting of a conformationally restricted nucleotide (CRN) monomer, a locked nucleic acid (LNA) monomer, a xylo-LNA monomer, an α-LNA monomer, an α-L-LNA monomer, a β-D-LNA monomer, a 2′-amino-LNA monomer, a 2′-(alkylamino)-LNA monomer, a 2′-(acylamino)-LNA monomer, a 2′-N-substituted-2′-amino-LNA monomer, a 2′-thio-LNA monomer, a (2′-O,4′-C) constrained ethyl (cEt) BNA monomer, a (2′-O,4′-C) constrained methoxyethyl (cMOE) BNA monomer, a 2′,4′-BNA^{NC}(N—H) monomer, a 2′,4′-BNA^{NC}(N-Me) monomer, a 2′,4′-BNA^{NC}(N-Bn) monomer, an ethylene-bridged nucleic acid (ENA) monomer, a carba LNA (cLNA) monomer, a 3,4-dihydro-2H-pyran nucleic acid (DpNA) monomer, a 2′-C-bridged bicyclic nucleotide (CBBN) monomer, a heterocyclic-bridged BNA monomer, an amido-bridged BNA monomer, an urea-bridged BNA monomer, a sulfonamide-bridged BNA monomer, a bicyclic carbocyclic nucleotide monomer, a TriNA monomer, an α-L-TriNA monomer, a bicyclo DNA (bcDNA) monomer, an F-bcDNA monomer, a tricyclo DNA (tcDNA) monomer, an F-tcDNA monomer, an oxetane nucleotide monomer, a locked PMO monomer derived from 2′-amino-LNA, and derivatives thereof. In one embodiment, more than one distinct scaffold BNA modification can be used in said oligonucleotide. In some embodiments, each occurrence of said BNA scaffold modification results in a monomer that is independently chosen from the group consisting of a conformationally restrained nucleotide (CRN) monomer, a locked nucleic acid (LNA) monomer, a xylo-LNA monomer, an α-L-LNA monomer, a β-D-LNA monomer, a 2′-amino-LNA monomer, a 2′-(alkylamino)-LNA monomer, a 2′-(acylamino)-LNA monomer, a 2′-N-substituted-2′-amino-LNA monomer, a (2′-O,4′-C) constrained ethyl (cEt) LNA monomer, a (2′-O,4′-C) constrained methoxyethyl (cMOE) BNA monomer, a 2′,4′-BNA^{NC}(N—H) monomer, a 2′,4′-BNA^{NC}(N-Me) monomer, an ethylene-bridged nucleic acid (ENA) monomer, a 2′-C-bridged bicyclic nucleotide (CBBN) monomer, and derivatives thereof. In one embodiment, each occurrence of said BNA scaffold modification is a locked nucleic acid (LNA) monomer.

Structural examples of monomers comprising these BNA scaffold modifications are shown below, where B is a base as defined herein, X is a variable and represents O, S or NR, where R is H or alkyl, X2 is a hydroxyl moiety or another 2′-substitution as defined herein, and L is a backbone linkage as described herein. As known to those of skill in the art, the naming of such modifications in the literature is often arbitrary and does not follow a uniform convention—in this application, the names as provided below are intended to refer to the structures provided below. For comparison, the cyclic scaffold of a conventional RNA monomer is shown first. In the structures shown below, monomers are typically depicted as 3′-terminal monomers. When chirality is not indicated, each enantiomer is individually referenced. A monomer resulting from the occurrence of a BNA scaffold modification in said antisense oligonucleotide of a hydroxyalkoxylated AON as described herein is not limited to this kind of monomers which are provided for illustrative purposes. Heteroatoms comprised in a cyclic moiety can be substituted by other heteroatoms, e.g., N, O or S.

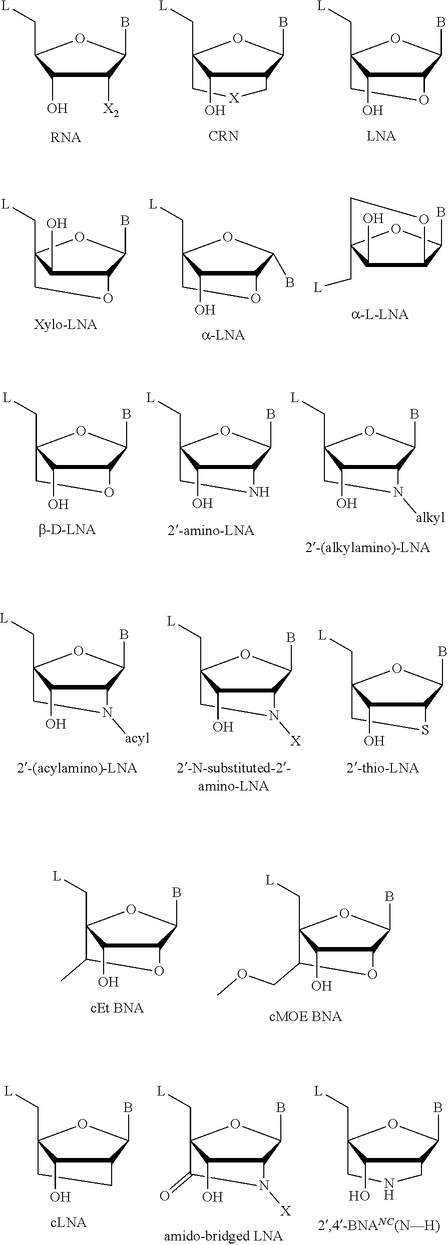

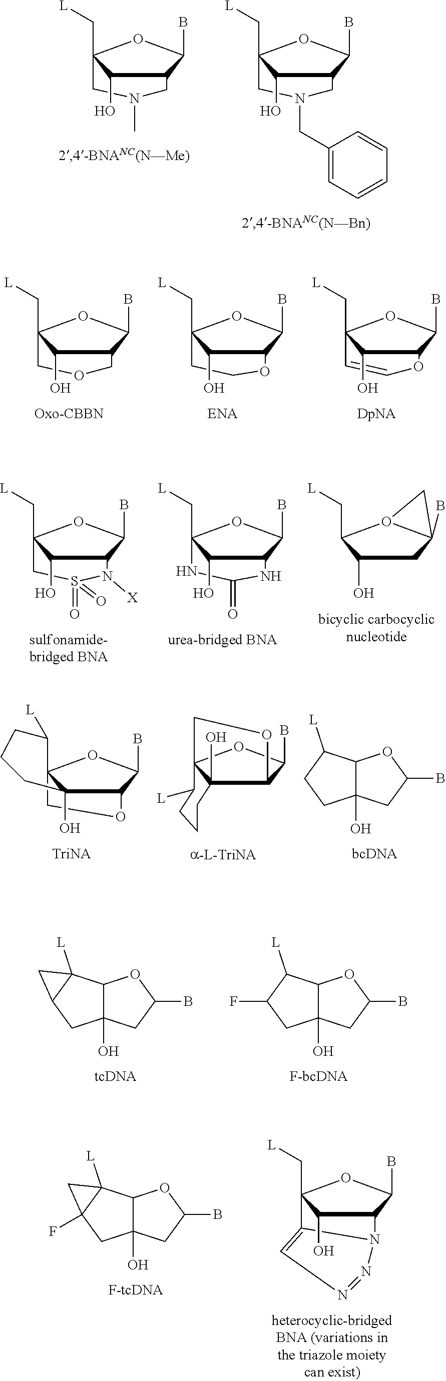

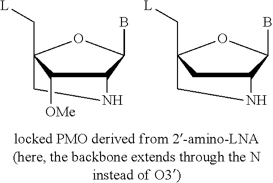

In another embodiment, BNA scaffold modifications for use herein include cEt (2′-O,4′-C constrained ethyl) LNA (doi: 10.1021/ja710342q), cMOE (2′-O,4′-C constrained methoxyethyl) LNA (Seth et al., J. Org. Chem. 2010, 75, 1569-1581), 2′,4′-BNA^{NC}(N—H), 2′,4′-BNA^{NC}(N-Me), ethylene-bridged nucleic acid (ENA) (doi: 10.1093/nass/1.1.241), carba LNA (cLNA) (doi: 10.1021/jo100170g), DpNA (Osawa et al., J. Org. Chem., 2015, 80 (21), pp 10474-10481), 2′-C-bridged bicyclic nucleotide (CBBN, as in e.g. WO 2014/145356 (MiRagen Therapeutics)), heterocyclic-bridged LNA (as in e.g. WO 2014/126229 (Mitsuoka Y et al.)), amido-bridged LNA (as in e.g. Yamamoto et al. Org. Biomol. Chem. 2015, 13, 3757), urea-bridged LNA (as in e.g. Nishida et al. Chem. Commun. 2010, 46, 5283), sulfonamide-bridged LNA (as in e.g. WO 2014/112463 (Obika S et al.)), bicyclic carbocyclic nucleosides (as in e.g. WO 2015/142910 (Ions Pharmaceuticals)), TriNA (Hanessian et al., J. Org. Chem., 2013, 78 (18), pp 9064-9075), α-L-TriNA, bicyclo DNA (bcDNA) (Bolli et al., Chem Biol. 1996 March; 3(3):197-206), F-bcDNA (DOI: 10.1021/jo402690j), tricyclo DNA (tcDNA) (Murray et al., Nucl. Acids Res., 2012, Vol. 40, No. 13 6135-6143), F-tcDNA (doi: 10.1021/acs.joc.5b00184), or an oxetane nucleotide monomer (Nucleic Acids Res. 2004, 32, 5791-5799). In other embodiments, BNA scaffold modifications for use herein include those disclosed in WO 2011/097641 (ISIS/Ionis Pharmaceuticals) and WO 2016/017422 (Osaka University).

In one embodiment, the antisense oligonucleotide of the hydroxyalkoxylated AON provided herein comprises RNA monomers. In one embodiment, an RNA oligonucleotide comprises a modification providing the RNA with an additional property, for instance resistance to endonucleases, exonucleases, and RNaseH, additional hybridisation strength, increased stability (for instance in a bodily fluid), increased or decreased flexibility, increased activity, reduced toxicity, increased intracellular transport, increased cellular uptake, or tissue-specificity, etc. In one embodiment, the mRNA complexed with said oligonucleotide is resistant to RNaseH cleavage.

In one embodiment, provided is a hydroxyalkoxylated AON that consists of one antisense oligonucleotide (AON) and a hydroxyalkoxy group (as described in the section entitled “Hydroxyalkoxy Groups”), said hydroxyalkoxy group is a triethylene glycol (TEG) group, wherein said antisense oligonucleotide is represented by a nucleotide sequence as defined herein (as described in the sections entitled “Hydroxyalkoxylated AON” and “Antisense oligonucleotide of the hydroxyalkoxylated AON”), and wherein said antisense oligonucleotide comprises 2′-O-methyl phosphorothioate RNA monomers linked by phosphorothioate backbone linkages, said AON further comprises at least one BNA, and optionally at least one 5-methylpyrimidine base (i.e., 5-methylcytosine and/or 5-methyluracil) is present. Throughout this specification, reference to an antisense oligonucleotide comprising 2′-O-methyl (phosphorothioate) RNA monomers and further comprising a BNA means that there is at least one BNA in the AON with the remainder of the monomers (non-BNA) in the AON being 2′-O-methyl (phosphorothioate) RNA monomers. In one embodiment, all cytosine bases of said AON are 5-methylcytosine bases. In another embodiment, at least one but less than all cytosine bases of said AON are 5-methylcytosine bases. In another embodiment, 1, 2, 3, 4, 5, 6, 7, 8 or 9 cytosine bases of said AON are 5-methyl cytosine bases. In one embodiment, said AON of the hydroxyalkoxylated AON comprises 2′-O-methyl RNA monomers connected through a phosphorothioate backbone and all of its cytosines have been substituted by 5-methylcytosine, optionally also all of its uracils have been substituted by 5-methyluracil, and at least one 2′-O-methyl scaffold has been replaced by a BNA, or 1, 2, 3, 4, 5, 6, 7, 8 or 9 monomers are replaced by a BNA. In one embodiment, 1, 2, 3, 4, 5, 6, 7, 8 or 9 monomers are replaced by a bridged nucleic acid scaffold modification. In one embodiment, 1, 2, 3, 4, 5, 6, 7, 8 or 9 monomers are replaced by a LNA.

In one embodiment, provided is a hydroxyalkoxylated AON, wherein the antisense oligonucleotide of said hydroxyalkoxylated AON comprises 2′-O-substituted RNA monomers linked by phosphorothioate backbone linkages and comprises or consists of 1, 2, 3, 4, 5, 6, 7, 8 or 9 monomers that comprise a bicyclic nucleic acid (BNA) scaffold modification, a bridged nucleic acid scaffold modification, or a LNA modification, and wherein all cytosine bases are 5-methylcytosine, wherein also all uracil bases are 5-methyluracil bases in said AON of the hydroxyalkoxylated AON as described herein.

In other embodiments, at least one BNA scaffold modification is comprised in a terminal monomer of said antisense oligonucleotide of the hydroxyalkoxylated AON. In one embodiment, such modification is in the 5′-terminal monomer. In another embodiment, both terminal monomers comprise a BNA scaffold. In one embodiment, provided is an antisense oligonucleotide (AON) of the hydroxyalkoxylated AON wherein at least one bicyclic nucleic acid (BNA) scaffold modification is comprised in a terminal monomer of said AON. In one embodiment, such BNA modification is in the 5′-terminal monomer of said AON, or in both terminal monomers of said AON. In some embodiments, a terminal monomer and its neighboring monomer each comprise a BNA scaffold. In such a case, the first two monomers and/or the last two monomers of said antisense oligonucleotide of the hydroxyalkoxylated AON each comprise a BNA scaffold. This can be combined in any way, so that for example the first and the last two monomers, or the first two and the last monomer all comprise a BNA scaffold. When the antisense oligonucleotide of a hydroxyalkoxylated AON described herein comprises a terminal monomer comprising a BNA scaffold, additional monomers with a BNA scaffold are either at the other terminus, or adjacent to terminal monomers with a BNA scaffold.

In one embodiment, provided is a hydroxyalkoxylated AON wherein the antisense oligonucleotide of said hydroxyalkoxylated AON, comprises or consists of BNA modifications as selected from the set consisting of:
a single BNA scaffold modification in the monomer at the 5′-terminus,
a single BNA scaffold modification in the monomer at the 3′-terminus,
two BNA scaffold modifications where one is in the monomer at the 5′-terminus and the other is in the monomer at the 3′-terminus,
two BNA scaffold modifications, one in the monomer at the 5′-terminus and the other in the adjacent monomer,
two BNA scaffold modifications, one in the monomer at the 3′-terminus and the other in the adjacent monomer, and
four BNA scaffold modifications, one in the monomer at the 5′-terminus, one in the monomer adjacent to the 5′-terminus, one in the monomer at the 3′-terminus and one in the monomer adjacent to the 3′-terminus;

optionally 1, 2, 3, 4 or 5 additional BNA scaffold modifications are present, wherein said antisense oligonucleotide is represented by a nucleotide sequence as defined herein and wherein said antisense oligonucleotide of the hydroxyalkoxylated AON consists of 2′-O-substituted RNA monomers linked by phosphorothioate backbone linkages, wherein all cytosine bases are 5-methylcytosine, optionally wherein also all uracil bases are 5-methyluracil bases in said AON of the hydroxyalkoxylated AON as described herein.

In one embodiment, antisense oligonucleotides of the hydroxyalkoxylated AON provided herein can be represented by any one of SEQ ID NO: 3177-3572 (derived from any one of SEQ ID NO: 9-404), or any one of SEQ ID NO: 3177-3179 (derived from any one of SEQ ID NO: 9-11), wherein said antisense oligonucleotide comprises a single BNA scaffold modification in the monomer at the 5′ terminus.

In one embodiment, antisense oligonucleotides of the hydroxyalkoxylated AON provided herein can be represented by any one of SEQ ID NO: 12681-13076 (derived from any one of SEQ ID NO: 9-404 respectively), or any one of SEQ ID NO: 12681-12683 (derived from any one of SEQ ID NO: 9-11 respectively), wherein said antisense oligonucleotide comprises a single BNA scaffold modification in the monomer at the 5′ terminus and wherein all cytosine bases are 5-methylcytosine bases.

In one embodiment, provided is a hydroxyalkoxylated AON for skipping exon 51 of the pre-mRNA of dystrophin, consisting or consisting essentially of one antisense oligonucleotide and one or two hydroxyalkoxy groups (as described in the section “Hydroxyalkoxy Groups”), wherein said antisense oligonucleotide is represented by a nucleotide sequence comprising or consisting of any one of SEQ ID NO: 3177-3572 (derived from any one of SEQ ID NO: 9-404, wherein a single BNA scaffold modification is present in the monomer at the 5′ terminus), or any one of SEQ ID NO: 3177-3179 (derived from any one of SEQ ID NO: 9-11, wherein a single BNA scaffold modification is present in the monomer at the 5′ terminus), and wherein said hydroxyalkoxylated AON can be represented by:

any one of SEQ ID NO: 3573-3968, or any one of SEQ ID NO: 3573-3575, if a hydroxyalkoxy group is present at the 5′ terminal monomer of the antisense oligonucleotide; or

any one of SEQ ID NO: 3969-4364, or any one of SEQ ID NO: 3969-3971, if a hydroxyalkoxy group is present at the 3′ terminal monomer of the antisense oligonucleotide; or

any one of SEQ ID NO: 4365-4760, or any one of SEQ ID NO: 4365-4367, if a first hydroxyalkoxy group is present at the 5′ terminal monomer and a second hydroxyalkoxy group is present at the 3′ terminal monomer of the antisense oligonucleotide;

wherein said hydroxyalkoxy group, represented by n in the sequence listing, is a triethylene glycol (TEG) group.

In one embodiment, provided is a hydroxyalkoxylated AON for skipping exon 51 of the pre-mRNA of dystrophin, consisting or consisting essentially of one antisense oligonucleotide and one or two hydroxyalkoxy group (as described in the section “Hydroxyalkoxy Groups”), wherein said antisense oligonucleotide is represented by a nucleotide sequence comprising or consisting of any one of SEQ ID NO: 12681-13076 (derived from any one of SEQ ID NO: 9-404, wherein a single BNA scaffold modification is present in the monomer at the 5′ terminus, and wherein all cytosine bases are 5-methylcytosine bases), or any one of SEQ ID NO: 12681-12683 (derived from any one of SEQ ID NO: 9-11, wherein a single BNA scaffold modification is present in the monomer at the 5′ terminus, and wherein all cytosine bases are 5-methylcytosine bases), and wherein said hydroxyalkoxylated AON can be represented by:

any one of SEQ ID NO: 13077-13472, or any one of SEQ ID NO: 13077-13079, if a hydroxyalkoxy group is present at the 5′ terminal monomer of the antisense oligonucleotide; or

any one of SEQ ID NO: 13473-13868, or any one of SEQ ID NO: 13473-13475, if a hydroxyalkoxy group is present at the 3′ terminal monomer of the antisense oligonucleotide; or

any one of SEQ ID NO: 13869-14264, or any one of SEQ ID NO: 13869-13871, if a first hydroxyalkoxy group is present at the 5′ terminal monomer and a second hydroxyalkoxy group is present at the 3′ terminal monomer of the antisense oligonucleotide;

wherein said hydroxyalkoxy group, represented by n in the sequence listing, is a triethylene glycol (TEG) group.

In one embodiment, oligonucleotides of the hydroxyalkoxylated AON provided herein can be represented by any one of SEQ ID NO: 4761-5156 (derived from any one of SEQ ID NO: 9-404 respectively), or any one of SEQ ID NO: 4761-4763 (derived from any one of SEQ ID NO: 9-11 respectively), wherein said antisense oligonucleotide comprises a single BNA scaffold modification in the monomer at the 3′ terminus.

In one embodiment, antisense oligonucleotides of the hydroxyalkoxylated AON provided herein can be represented by any one of SEQ ID NO: 14265-14660 (derived from any one of SEQ ID NO: 9-404 respectively), or any one of SEQ ID NO: 14265-14267 (derived from any one of SEQ ID NO: 9-11 respectively), wherein said antisense oligonucleotide comprises a single BNA scaffold modification in the monomer at the 3′ terminus and wherein all cytosine bases are 5-methylcytosine bases.

In one embodiment is provided a hydroxyalkoxylated AON for skipping exon 51 of the pre-mRNA of dystrophin, consisting or consisting essentially of one antisense oligonucleotide and one or two hydroxyalkoxy groups (as described in the section “Hydroxyalkoxy Groups”), wherein said antisense oligonucleotide is represented by a nucleotide sequence comprising or consisting of any one of SEQ ID NO: 4761-5156 (derived from any one of SEQ ID NO: 9-404, wherein a single BNA scaffold modification is present in the monomer at the 3′ terminus), or any one of SEQ ID NO: 4761-4763 (derived from any one of SEQ ID NO: 9-11, wherein a single BNA scaffold modification is present in the monomer at the 3′ terminus), and wherein said hydroxyalkoxylated AON can be represented by:

any one of SEQ ID NO: 5157-5552, or any one of SEQ ID NO: 5157-5159, if a hydroxyalkoxy group is present at the 5′ terminal monomer of the antisense oligonucleotide; or

any one of SEQ ID NO: 5553-5948, or any one of SEQ ID NO: 5553-5555, if a hydroxyalkoxy group is present at the 3′ terminal monomer of the antisense oligonucleotide; or

any one of SEQ ID NO: 5949-6344, or any one of SEQ ID NO: 5949-5951, if a first hydroxyalkoxy group is present at the 5′ terminal monomer and a second hydroxyalkoxy group is present at the 3′ terminal monomer of the antisense oligonucleotide;

wherein said hydroxyalkoxy group, represented by n in the sequence listing, is a triethylene glycol (TEG) group.

In one embodiment is provided a hydroxyalkoxylated AON for skipping exon 51 of the pre-mRNA of dystrophin, consisting or consisting essentially of one antisense oligonucleotide and one or two hydroxyalkoxy group (as described in the section “Hydroxyalkoxy Groups”), wherein said antisense oligonucleotide is represented by a nucleotide sequence comprising or consisting of any one of SEQ ID NO: 14265-14660 (derived from any one of SEQ ID NO: 9-404, wherein a single BNA scaffold modification is present in the monomer at the 3′ terminus, and wherein all cytosine bases are 5-methylcytosine bases), or any one of SEQ ID NO: 14265-14267 (derived from any one of SEQ ID NO: 9-11, wherein a single BNA scaffold modification is present in the monomer at the 3′ terminus, and wherein all cytosine bases are 5-methylcytosine bases), and wherein said hydroxyalkoxylated AON can be represented by:

any one of SEQ ID NO: 14661-15056, or any one of SEQ ID NO: 14661-14663, if a hydroxyalkoxy group is present at the 5′ terminal monomer of the antisense oligonucleotide; or

any one of SEQ ID NO: 15057-15452, or any one of SEQ ID NO: 15057-15059, if a hydroxyalkoxy group is present at the 3′ terminal monomer of the antisense oligonucleotide; or

any one of SEQ ID NO: 15453-15848, or any one of SEQ ID NO: 15453-15455, if a first hydroxyalkoxy group is present at the 5′ terminal monomer and a second hydroxyalkoxy group is present at the 3′ terminal monomer of the antisense oligonucleotide;

wherein said hydroxyalkoxy group, represented by n in the sequence listing, is a triethylene glycol (TEG) group.

In one embodiment, antisense oligonucleotides of the hydroxyalkoxylated AON provided herein can be represented by any one of SEQ ID NO: 6345-6740 (derived from any one of SEQ ID NO: 9-404 respectively), or any one of SEQ ID NO: 6345-6347 (derived from any one of SEQ ID NO: 9-11 respectively), wherein said antisense oligonucleotide comprises a single BNA scaffold modification in the monomer at the 5′ terminus and a single BNA scaffold modification in the monomer at the 3′ terminus.

In one embodiment, antisense oligonucleotides of the hydroxyalkoxylated AON described herein can be represented by any one of SEQ ID NO: 15849-16244 (derived from any one of SEQ ID NO: 9-404 respectively), or any one of SEQ ID NO: 15849-15851 (derived from any one of SEQ ID NO: 9-11 respectively), wherein said antisense oligonucleotide comprises a single BNA scaffold modification in the monomer at the 5′ terminus and a single BNA scaffold modification in the monomer at the 3′ terminus, and wherein all cytosine bases are 5-methylcytosine bases.

In one embodiment provided is a hydroxyalkoxylated AON for skipping exon 51 of the pre-mRNA of dystrophin, consisting or consisting essentially of one antisense oligonucleotide and one or two hydroxyalkoxy groups (as described in the section “Hydroxyalkoxy Groups”), wherein said antisense oligonucleotide is represented by a nucleotide sequence comprising or consisting of any one of SEQ ID NO: 6345-6740 (derived from any one of SEQ ID NO: 9-404, wherein a single BNA scaffold modification is present in the monomer at the 5′ terminus and a single BNA scaffold modification is present in the monomer at the 3′ terminus), or any one of SEQ ID NO: 6345-6347 (derived from any one of SEQ ID NO: 9-11, wherein a single BNA scaffold modification is present in the monomer at the 5′ terminus and a single BNA scaffold modification is present in the monomer at the 3′ terminus), and wherein said hydroxyalkoxylated AON can be represented by:

any one of SEQ ID NO: 6741-7136, or any one of SEQ ID NO: 6741-6743, if a hydroxyalkoxy group is present at the 5′ terminal monomer of the antisense oligonucleotide; or

any one of SEQ ID NO: 7137-7532, or any one of SEQ ID NO: 7137-7139, if a hydroxyalkoxy group is present at the 3′ terminal monomer of the antisense oligonucleotide; or

any one of SEQ ID NO: 7533-7928, or any one of SEQ ID NO: 7533-7535, if a first hydroxyalkoxy group is present at the 5′ terminal monomer and a second hydroxyalkoxy group is present at the 3′ terminal monomer of the antisense oligonucleotide;

wherein said hydroxyalkoxy group, represented by n in the sequence listing, is a triethylene glycol (TEG) group.

In one embodiment provided is a hydroxyalkoxylated AON for skipping exon 51 of the pre-mRNA of dystrophin, consisting or consisting essentially of one antisense oligonucleotide and one or two hydroxyalkoxy groups (as described in the section “Hydroxyalkoxy Groups”), wherein said antisense oligonucleotide is represented by a nucleotide sequence comprising or consisting of any one of SEQ ID NO: 15849-16244 (derived from any one of SEQ ID NO: 9-404, wherein a single BNA scaffold modification is present in the monomer at the 5′ terminus and a single BNA scaffold modification is present in the monomer at the 3′ terminus, and wherein all cytosine bases are 5-methylcytosine bases), or any one of SEQ ID NO: 15849-15851 (derived from any one of SEQ ID NO: 9-11, wherein a single BNA scaffold modification is present in the monomer at the 5′ terminus and a single BNA scaffold modification is present in the monomer at the 3′ terminus, and wherein all cytosine bases are 5-methylcytosine bases), and wherein said hydroxyalkoxylated AON can be represented by:

any one of SEQ ID NO: 16245-16640, or any one of SEQ ID NO: 16245-16247, if a hydroxyalkoxy group is present at the 5′ terminal monomer of the antisense oligonucleotide; or

any one of SEQ ID NO: 16641-17036, or any one of SEQ ID NO: 16641-16643, if a hydroxyalkoxy group is present at the 3′ terminal monomer of the antisense oligonucleotide; or

any one of SEQ ID NO: 17037-17432, or any one of SEQ ID NO: 17037-17039, if a first hydroxyalkoxy group is present at the 5′ terminal monomer and a second hydroxyalkoxy group is present at the 3′ terminal monomer of the antisense oligonucleotide;

wherein said hydroxyalkoxy group, represented by n in the sequence listing, is a triethylene glycol (TEG) group.

In one embodiment, antisense oligonucleotides of the hydroxyalkoxylated AON provided herein can be represented by any one of SEQ ID NO: 7929-8324 (derived from any one of SEQ ID NO: 9-404 respectively), or any one of SEQ ID NO: 7929-7931 (derived from any one of SEQ ID NO: 9-11 respectively), wherein said antisense oligonucleotide comprises two BNA scaffold modifications, one in each of the two monomers that are “-terminus.

In one embodiment, antisense oligonucleotides of the hydroxyalkoxylated AON described herein can be represented by any one of SEQ ID NO: 17433-17828 (derived from any one of SEQ ID NO: 9-404 respectively), or any one of SEQ ID NO: 17433-17435 (derived from any one of SEQ ID NO: 9-11 respectively), wherein said antisense oligonucleotide comprises two BNA scaffold modifications, one in the monomer at the 5′-terminus and the other in the adjacent monomer, and wherein all cytosine bases are 5-methylcytosine bases.

In one embodiment, provided is a hydroxyalkoxylated AON for skipping exon 51 of the pre-mRNA of dystrophin, consisting or consisting essentially of one antisense oligonucleotide and one or two hydroxyalkoxy groups (as described in the section “Hydroxyalkoxy Groups”), wherein said antisense oligonucleotide is represented by a nucleotide sequence comprising or consisting of any one of SEQ ID NO: 7929-8324 (derived from any one of SEQ ID NO: 9-404, wherein two BNA scaffold modifications are present, one in the monomer at the 5′-terminus and the other in the adjacent monomer), or any one of SEQ ID NO: 7929-7931 (derived from any one of SEQ ID NO: 9-11, wherein two BNA scaffold modifications are present, one in the monomer at the 5′-terminus and the other in the adjacent monomer), and wherein said hydroxyalkoxylated AON can be represented by:

any one of SEQ ID NO: 8325-8720, or any one of SEQ ID NO: 8325-8327, if a hydroxyalkoxy group is present at the 5′ terminal monomer of the antisense oligonucleotide; or

any one of SEQ ID NO: 8721-9116, or any one of SEQ ID NO: 8721-8723, if a hydroxyalkoxy group is present at the 3′ terminal monomer of the antisense oligonucleotide; or

any one of SEQ ID NO: 9117-9512, or any one of SEQ ID NO: 9117-9119, if a first hydroxyalkoxy group is present at the 5′ terminal monomer and a second hydroxyalkoxy group is present at the 3′ terminal monomer of the antisense oligonucleotide;

wherein said hydroxyalkoxy group, represented by n in the sequence listing, is a triethylene glycol (TEG) group.

In one embodiment is provided a hydroxyalkoxylated AON for skipping exon 51 of the pre-mRNA of dystrophin, consisting or consisting essentially of one antisense oligonucleotide and one or two hydroxyalkoxy groups (as described in the section “Hydroxyalkoxy Groups”), or one or two hydroxyalkoxy groups, wherein said antisense oligonucleotide is represented by a nucleotide sequence comprising or consisting of any one of SEQ ID NO: 17433-17828 (derived from any one of SEQ ID NO: 9-404, wherein two BNA scaffold modifications are present, one in the monomer at the 5′-terminus and the other in the adjacent monomer, and wherein all cytosine bases are 5-methylcytosine bases), or any one of SEQ ID NO: 17433-17435 (derived from any one of SEQ ID NO: 9-11, wherein two BNA scaffold modifications are present, one in the monomer at the 5′-terminus and the other in the adjacent monomer, and wherein all cytosine bases are 5-methylcytosine bases), and wherein said hydroxyalkoxylated AON can be represented by:

any one of SEQ ID NO: 17829-18224, or any one of SEQ ID NO: 17829-17831, if a hydroxyalkoxy group is present at the 5′ terminal monomer of the antisense oligonucleotide; or

any one of SEQ ID NO: 18225-18620, or any one of SEQ ID NO: 18225-18227, if a hydroxyalkoxy group is present at the 3′ terminal monomer of the antisense oligonucleotide; or

any one of SEQ ID NO: 18621-19016, or any one of SEQ ID NO: 18621-18623, if a first hydroxyalkoxy group is present at the 5′ terminal monomer and a second hydroxyalkoxy group is present at the 3′ terminal monomer of the antisense oligonucleotide;

wherein said hydroxyalkoxy group, represented by n in the sequence listing, is a triethylene glycol (TEG) group.

In one embodiment, antisense oligonucleotides of the hydroxyalkoxylated AON provided herein can be represented by any one of SEQ ID NO: 9513-9908 (derived from any one of SEQ ID NO: 9-404 respectively), or any one of SEQ ID NO: 9513-9515 (derived from any one of SEQ ID NO: 9-11 respectively), wherein said antisense oligonucleotide comprises two BNA scaffold modifications, one in the monomer at the 3′-terminus and the other in the adjacent monomer.

In one embodiment, the antisense oligonucleotides of the hydroxyalkoxylated AON provided herein can be represented by any one of SEQ ID NO: 19017-19412 (derived from any one of SEQ ID NO: 9-404 respectively), or any one of SEQ ID NO: 19017-19019 (derived from any one of SEQ ID NO: 9-11 respectively), wherein said antisense oligonucleotide comprises two BNA scaffold modifications, one in the monomer at the 3′-terminus and the other in the adjacent monomer, and wherein all cytosine bases are 5-methylcytosine bases.

In one embodiment is provided a hydroxyalkoxylated AON for skipping exon 51 of the pre-mRNA of dystrophin, consisting or consisting essentially of one antisense oligonucleotide and one or two hydroxyalkoxy groups (as described in the section “Hydroxyalkoxy Groups”), wherein said antisense oligonucleotide is represented by a nucleotide sequence comprising or consisting of any one of SEQ ID NO: 9513-9908 (derived from any one of SEQ ID NO: 9-404, wherein two BNA scaffold modifications are present, one in the monomer at the 3′-terminus and the other in the adjacent monomer), or any one of SEQ ID NO: 9513-9515 (derived from any one of SEQ ID NO: 9-11, wherein two BNA scaffold modifications are present, one in the monomer at the 3′-terminus and the other in the adjacent monomer), and wherein said hydroxyalkoxylated AON can be represented by:

any one of SEQ ID NO: 9909-10304, or any one of SEQ ID NO: 9909-9911, if a hydroxyalkoxy group is present at the 5′ terminal monomer of the antisense oligonucleotide; or

any one of SEQ ID NO: 10305-10700, or any one of SEQ ID NO: 10305-10307, if a hydroxyalkoxy group is present at the 3′ terminal monomer of the antisense oligonucleotide; or

any one of SEQ ID NO: 10701-11096, or any one of SEQ ID NO: 10701-10703, if a first hydroxyalkoxy group is present at the 5′ terminal monomer and a second hydroxyalkoxy group is present at the 3′ terminal monomer of the antisense oligonucleotide;

wherein said hydroxyalkoxy group, represented by n in the sequence listing, is a triethylene glycol (TEG) group.

In one embodiment is provided a hydroxyalkoxylated AON for skipping exon 51 of the pre-mRNA of dystrophin, consisting or consisting essentially of one antisense oligonucleotide and one or two hydroxyalkoxy groups (as described in the section “Hydroxyalkoxy Groups”), wherein said antisense oligonucleotide is represented by a nucleotide sequence comprising or consisting of any one of SEQ ID NO: 19017-19412 (derived from any one of SEQ ID NO: 9-404, wherein two BNA scaffold modifications are present, one in the monomer at the 3′-terminus and the other in the adjacent monomer, and wherein all cytosine bases are 5-methylcytosine bases), or any one of SEQ ID NO: 19017-19019 (derived from any one of SEQ ID NO: 9-11, wherein two BNA scaffold modifications are present, one in the monomer at the 3′-terminus and the other in the adjacent monomer, and wherein all cytosine bases are 5-methylcytosine bases), and wherein said hydroxyalkoxylated AON can be represented by:

any one of SEQ ID NO: 19413-19808, or any one of SEQ ID NO: 19413-19415, if a hydroxyalkoxy group is present at the 5′ terminal monomer of the antisense oligonucleotide; or

any one of SEQ ID NO: 19809-20204, or any one of SEQ ID NO: 19809-19811, if a hydroxyalkoxy group is present at the 3′ terminal monomer of the antisense oligonucleotide; or

any one of SEQ ID NO: 20205-20600, or any one of SEQ ID NO: 20205-20207, if a first hydroxyalkoxy group is present at the 5′ terminal monomer and a second hydroxyalkoxy group is present at the 3′ terminal monomer of the antisense oligonucleotide;

wherein said hydroxyalkoxy group, represented by n in the sequence listing, is a triethylene glycol (TEG) group.

In one embodiment, antisense oligonucleotides of the hydroxyalkoxylated AON provided herein can be represented by any one of SEQ ID NO: 11097-11492 (derived from any one of SEQ ID NO: 9-404 respectively), or any one of SEQ ID NO: 11097-11099 (derived from any one of SEQ ID NO: 9-11 respectively), wherein said antisense oligonucleotide comprises four BNA scaffold modifications, one in the monomer at the 5′-terminus, one in the monomer adjacent to the 5′-terminus, one in the monomer at the 3′-terminus and one in the monomer adjacent to the 3′-terminus.

In one embodiment, antisense oligonucleotides of the hydroxyalkoxylated AON provided herein can be represented by any one of SEQ ID NO: 20601-20996 (derived from any one of SEQ ID NO: 9-404 respectively), or any one of SEQ ID NO: 20601-20603 (derived from any one of SEQ ID NO: 9-11 respectively), wherein said antisense oligonucleotide comprises four BNA scaffold modifications, one in the monomer at the 5′-terminus, one in the monomer adjacent to the 5′-terminus, one in the monomer at the 3′-terminus and one in the monomer adjacent to the 3′-terminus, and wherein all cytosine bases are 5-methylcytosine bases.

In one embodiment is provided a hydroxyalkoxylated AON for skipping exon 51 of the pre-mRNA of dystrophin, consisting or consisting essentially of one antisense oligonucleotide and one or two hydroxyalkoxy groups (as described in the section “Hydroxyalkoxy Groups”), wherein said antisense oligonucleotide is represented by a nucleotide sequence comprising or consisting of any one of SEQ ID NO: 11097-11492 (derived from any one of SEQ ID NO: 9-404, wherein four BNA scaffold modifications are present, one in the monomer at the 5′-terminus, one in the monomer adjacent to the 5′-terminus, one in the monomer at the 3′-terminus and one in the monomer adjacent to the 3′-terminus), or any one of SEQ ID NO: 11097-11099 (derived from any one of SEQ ID NO: 9-11, wherein four BNA scaffold modifications are present, one in the monomer at the 5′-terminus, one in the monomer adjacent to the 5′-terminus, one in the monomer at the 3′-terminus and one in the monomer adjacent to the 3′-terminus), and wherein said hydroxyalkoxylated AON can be represented by:

any one of SEQ ID NO: 11493-11888, or any one of SEQ ID NO: 11493-11495, if a hydroxyalkoxy group is present at the 5′ terminal monomer of the antisense oligonucleotide; or

any one of SEQ ID NO: 11889-12284, or any one of SEQ ID NO: 11889-11891, if a hydroxyalkoxy group is present at the 3′ terminal monomer of the antisense oligonucleotide; or

any one of SEQ ID NO: 12285-12680, or any one of SEQ ID NO: 12285-12287, if a first hydroxyalkoxy group is present at the 5′ terminal monomer and a second hydroxyalkoxy group is present at the 3′ terminal monomer of the antisense oligonucleotide;

wherein said hydroxyalkoxy group, represented by n in the sequence listing, is a triethylene glycol (TEG) group.

In one embodiment is provided a hydroxyalkoxylated AON for skipping exon 51 of the pre-mRNA of dystrophin, consisting or consisting essentially of one antisense oligonucleotide and one or two hydroxyalkoxy groups (as described in the section “Hydroxyalkoxy Groups”), wherein said antisense oligonucleotide is represented by a nucleotide sequence comprising or consisting of any one of SEQ ID NO: 20601-20996 (derived from any one of SEQ ID NO: 9-404, wherein four BNA scaffold modifications are present, one in the monomer at the 5′-terminus, one in the monomer adjacent to the 5′-terminus, one in the monomer at the 3′-terminus and one in the monomer adjacent to the 3′-terminus, and wherein all cytosine bases are 5-methylcytosine bases), or any one of SEQ ID NO: 20601-20603 (derived from any one of SEQ ID NO: 9-11, wherein four BNA scaffold modifications are present, one in the monomer at the 5′-terminus, one in the monomer adjacent to the 5′-terminus, one in the monomer at the 3′-terminus and one in the monomer adjacent to the 3′-terminus, and wherein all cytosine bases are 5-methylcytosine bases), and wherein said hydroxyalkoxylated AON can be represented by:

any one of SEQ ID NO: 20997-21392, or any one of SEQ ID NO: 20997-20999, if a hydroxyalkoxy group is present at the 5′ terminal monomer of the antisense oligonucleotide; or

any one of SEQ ID NO: 21393-21788, or any one of SEQ ID NO: 21393-21395, if a hydroxyalkoxy group is present at the 3′ terminal monomer of the antisense oligonucleotide; or

any one of SEQ ID NO: 21789-22184, or any one of SEQ ID NO: 21789-21791, if a first hydroxyalkoxy group is present at the 5′ terminal monomer and a second hydroxyalkoxy group is present at the 3′ terminal monomer of the antisense oligonucleotide;

wherein said hydroxyalkoxy group, represented by n in the sequence listing, is a triethylene glycol (TEG) group.

Throughout this application, whenever a SEQ ID NO references T or U, said monomer can optionally be replaced by U or T, respectively.

The antisense oligonucleotide of the hydroxyalkoxylated AON described herein comprises terminal and non-terminal monomers. In the context of this application, terminal monomers are defined as monomers chosen from the group consisting of the 5′-terminal monomer and the 3′-terminal monomer of said antisense oligonucleotide, as explained herein. Non-terminal monomers of said antisense oligonucleotide are defined herein as monomers comprised in said antisense oligonucleotide which are not terminal monomers.

In one embodiment, both terminal and non-terminal monomers of the antisense oligonucleotide of the hydroxyalkoxylated AON may comprise a BNA scaffold modification.

In one embodiment, provided is a hydroxyalkoxylated AON consisting of one antisense oligonucleotide (AON) and one or two hydroxyalkoxy groups (as described in the section entitled “Hydroxyalkoxy Groups”), said hydroxyalkoxy group is a triethylene glycol (TEG) group, wherein said antisense oligonucleotide is represented by a nucleotide sequence as defined herein, wherein said AON comprises 2′-O-methyl phosphorothioate RNA monomers linked by phosphorothioate backbone and wherein said AON comprises or consists of 1, 2, 3, 4, 5, 6, 7, 8 or 9 monomers, or 1, 2, 3, 4 or 5 monomers, that comprise a bicyclic nucleic acid (BNA) scaffold modification, or a bridged nucleic acid scaffold modification, or a LNA modification, and wherein all cytosine bases are 5-methylcytosine bases, optionally wherein also all uracil bases are 5-methyluracil bases as defined herein.

In one embodiment, the antisense oligonucleotide (AON) of the hydroxyalkoxylated AON provided herein, is selected wherein:

the 5′-terminal monomer of said AON comprises a BNA scaffold modification, or

the 3′-terminal monomer of said AON comprises a BNA scaffold modification, or

the 5′-terminal monomer and the 3′-terminal monomer of said AON comprise a BNA scaffold modification, or

the two most 5′-terminal monomers of said AON comprise a BNA scaffold modification, or

the two most 3′ terminal monomers of said AON comprise a BNA scaffold modification, or

the two most 5′-terminal monomers and the two most 3′-terminal monomers of said AON comprise a BNA scaffold modification;

and wherein said AON comprises or consists of 1, 2, 3, 4 or 5 additional non-terminal monomers comprising a BNA scaffold modification, or 1 or 2 additional non-terminal monomers comprising a BNA scaffold modification, wherein said additional non-terminal monomers comprise an adenosine, a uracil and/or thymine base; or a guanine, a cytosine and/or a 5-methylcytosine base. In one embodiment, a BNA scaffold modification is an LNA modification.

In another embodiment, the antisense oligonucleotide of the hydroxyalkoxylated AON described herein is selected wherein said antisense oligonucleotide is represented by the nucleotide sequence GGUAAGUUCUGUCCAAGC (SEQ ID NO: 22185, derived from SEQ ID NO: 11), wherein G is a guanine comprising a BNA scaffold modification and wherein C is a cytosine comprising a BNA scaffold modification.

In another embodiment, the antisense oligonucleotide of the hydroxyalkoxylated AON described herein, is selected wherein said antisense oligonucleotide is represented by the nucleotide sequence GGUAAGUUC*UGUC*C*AAGC* (SEQ ID NO: 22333, derived from SEQ ID NO: 11), wherein G is a guanine comprising a BNA scaffold modification, C* is 5-methylcytosine and wherein C* is a 5-methylcytosine comprising a BNA scaffold modification.

In another embodiment is provided a hydroxyalkoxylated AON consisting or consisting essentially of one antisense oligonucleotide and one or two hydroxyalkoxy groups (as described in the section “Hydroxyalkoxy Groups”), said hydroxyalkoxy group comprises or consists of an ethylene glycol monomer, ethylene glycol oligomer or ethylene glycol polymer, or a triethylene glycol (TEG) group, wherein said antisense oligonucleotide is represented by a nucleotide sequence comprising or consisting of any one of:

SEQ ID NO: 22185,

a fragment of SEQ ID NO: 22185 or

SEQ ID NO: 22185 with 1, 2, 3, 4, or 5 additional nucleotides, or

SEQ ID NO: 22185 with 1, 2, 3, 4, or 5 nucleotides missing, or

a nucleotide sequence which has at least 90%, 91%, 92%, 93%, 94%, 95%, 96%, 97%, 98% or 99%, or at least 95%, more or at least 97%, identity with SEQ ID NO: 22185,

and wherein said AON consists of 2′-O-methyl RNA monomers linked by phosphorothioate backbone linkages, for use as a medicament, or for treating, preventing and/or delaying Duchenne Muscular Dystrophy (DMD), or for inducing skipping of exon 51 of the dystrophin pre-mRNA. In one embodiment, said exon 51 of dystrophin pre-mRNA is from a human and is represented by a nucleotide sequence with SEQ ID NO: 2.

In one embodiment, said 1, 2, 3, 4 or 5 additional nucleotides may be present at the 5′ and/or 3′ terminus of SEQ ID NO: 22185.

In another embodiment, said 1, 2, 3, 4 or 5 missing nucleotides may be nucleotides present at the 5′ and/or 3′ terminus of SEQ ID NO: 22185.

In another embodiment, provided is a hydroxyalkoxylated AON consisting or consisting essentially of one antisense oligonucleotide and one or two hydroxyalkoxy groups (as described in the section “Hydroxyalkoxy Groups”), said hydroxyalkoxy group comprises or consists of an ethylene glycol monomer, ethylene glycol oligomer or ethylene glycol polymer, or a triethylene glycol (TEG) group, wherein said antisense oligonucleotide is represented by a nucleotide sequence comprising or consisting of any one of:

SEQ ID NO: 22333,

a fragment of SEQ ID NO: 22333 or

SEQ ID NO: 22333 with 1, 2, 3, 4, or 5 additional nucleotides, or

SEQ ID NO: 22333 with 1, 2, 3, 4, or 5 nucleotides missing, or

a nucleotide sequence which has at least 90%, 91%, 92%, 93%, 94%, 95%, 96%, 97%, 98% or 99%, or at least 95%, or at least 97%, identity with SEQ ID NO: 22333,

and wherein said AON consists of 2′-O-methyl RNA monomers linked by phosphorothioate backbone linkages, for use as a medicament, or for treating, preventing and/or delaying Duchenne Muscular Dystrophy (DMD), or for inducing skipping of exon 51 of the dystrophin pre-mRNA. In one embodiment, said exon 51 of dystrophin pre-mRNA is from a human and is represented by a nucleotide sequence with SEQ ID NO: 2.

In one embodiment, said 1, 2, 3, 4 or 5 additional nucleotides may be present at the 5′ and/or 3′ terminus of SEQ ID NO: 22333.

In another embodiment, said 1, 2, 3, 4 or 5 missing nucleotides may be nucleotides present at the 5′ and/or 3′ terminus of SEQ ID NO: 22333.

In another embodiment, provided is a hydroxyalkoxylated AON consisting or consisting essentially of one antisense oligonucleotide and one or two hydroxyalkoxy groups (as described in the section “Hydroxyalkoxy Groups”), said hydroxyalkoxy group comprises or consists of an ethylene glycol monomer, ethylene glycol oligomer or ethylene glycol polymer, or a triethylene glycol (TEG) group, wherein said antisense oligonucleotide is represented by a nucleotide sequence comprising or consisting of any one of:

SEQ ID NO: 22486,

a fragment of SEQ ID NO: 22486 or

SEQ ID NO: 22486 with 1, 2, 3, 4, or 5 additional nucleotides, or

SEQ ID NO: 22486 with 1, 2, 3, 4, or 5 nucleotides missing, or

a nucleotide sequence which has at least 90%, 91%, 92%, 93%, 94%, 95%, 96%, 97%, 98% or 99%, or at least 95%, or at least 97%, identity with SEQ ID NO: 22486,

and wherein said AON consists of 2′-O-methyl RNA monomers linked by phosphorothioate backbone linkages, for use as a medicament, or for treating, preventing and/or delaying Duchenne Muscular Dystrophy (DMD), or for inducing skipping of exon 51 of the dystrophin pre-mRNA. In one embodiment, said exon 51 of dystrophin pre-mRNA is from a human and is represented by a nucleotide sequence with SEQ ID NO: 2.

In one embodiment, said 1, 2, 3, 4 or 5 additional nucleotides may be present at the 5′ and/or 3′ terminus of SEQ ID NO: 22486.

In another embodiment, said 1, 2, 3, 4 or 5 missing nucleotides may be nucleotides present at the 5′ and/or 3′ terminus of SEQ ID NO: 22486.

In one embodiment, hydroxyalkoxylated AONs can be represented by SEQ ID NO: 22345 (nGGUAAGUUC*UGUC*C*AAGC*, wherein a hydroxyalkoxy group is present at the 5′ terminal monomer), SEQ ID NO: 22357 (GGUAAGUUC*UGUC*C*AAGC*n, wherein a hydroxyalkoxy group is present at the 3′ terminal monomer), SEQ ID NO: 22369 (nGGUAAGUUC*UGUC*C*AAGC*n, wherein a first hydroxyalkoxy group is present at the 5′ terminal monomer and a second hydroxyalkoxy group is present at the 3′ terminal monomer), SEQ ID NO: 22488 (nGGUAAGUUC*UGUC*C*AAG, wherein a hydroxyalkoxy group is present at the 5′ terminal monomer), SEQ ID NO: 22489 (nGGUAAGUUC*UGUC*C*AA, wherein a hydroxyalkoxy group is present at the 5′ terminal monomer), or SEQ ID NO: 22490 (nGGUAAGUUC*UGUC*C*AAGC*, wherein a hydroxyalkoxy group is present at the 5′ terminal monomer and the non-BNA monomers are 2′-O-methoxyethyl monomers), wherein the hydroxyalkoxy group, represented by n, is a triethylene glycol (TEG) group, wherein G and C* are a guanine and a 5-methylcytosine, respectively, comprising a BNA scaffold modification and wherein C* is a 5-methylcytosine.

In some embodiments, the hydroxyalkoxylated AON consisting of one antisense oligonucleotide and one or two hydroxyalkoxy groups (as described in the section “Hydroxyalkoxy Groups”), is one wherein said hydroxyalkoxylated AON has an improved parameter by comparison to said antisense oligonucleotide alone and/or to a mixture of said antisense oligonucleotide and said hydroxyalkoxy group(s) where both antisense oligonucleotide and hydroxyalkoxy group(s) are present as separate molecules, i.e., not linked to each other by a covalent linkage, wherein the concentration of said antisense oligonucleotide and the hydroxyalkoxy group(s) is the same as in the hydroxyalkoxylated AON described herein. In another embodiment, the hydroxyalkoxylated AON is one wherein said hydroxyalkoxylated AON has an improved parameter by comparison to drisapersen (SEQ ID NO: 7, i.e., UCAAGGAAGAUGGCAUUUCU, wherein each RNA monomer is 2′-O-methylated and wherein the whole backbone is phosphorothioate), suvodirsen (WVE-210201) as described in WO 2017/062862, having the same sequence as drisapersen but with stereopure internucleoside linkages, and/or eteplirsen (SEQ ID NO: 8, i.e., CTCCAACATCAAGGAAGATGGCATTTCTAG, wherein each monomer is modified as to form a phosphorodiamidate morpholino oligomer).

In one embodiment, the hydroxyalkoxylated AON has higher compound stability than the non-hydroxyalkoxylated AON. In one embodiment, the hydroxyalkoxylated AON has better bio-distribution than the non-hydroxyalkoxylated AON. In one embodiment, the hydroxyalkoxylated AON has better muscle uptake than the non-hydroxyalkoxylated AON. In one embodiment, the hydroxyalkoxylated AON has better quadriceps muscle uptake than the non-hydroxyalkoxylated AON. In one embodiment, the hydroxyalkoxylated AON has better heart muscle uptake than the non-hydroxyalkoxylated AON. In one embodiment, the hydroxyalkoxylated AON has more efficient nuclear trafficking than the non-hydroxyalkoxylated AON. In one embodiment, the hydroxyalkoxylated AON has a better hindrance of splicing factors than the non-hydroxyalkoxylated AON.

In one embodiment, the hydroxyalkoxylated AON has higher binding affinity than the non-hydroxyalkoxylated AON. In one embodiment, the hydroxyalkoxylated AON has better kinetics than the non-hydroxyalkoxylated AON. In one embodiment, the hydroxyalkoxylated AON has improved exon skipping activity than the non-hydroxyalkoxylated AON. In one embodiment, the hydroxyalkoxylated AON provides a subject with a functional or a semi-functional dystrophin protein. In one embodiment, the hydroxyalkoxylated AON has improved biostability than the non-hydroxyalkoxylated AON. In one embodiment, the hydroxyalkoxylated AON has improved (intra-tissue) distribution than the non-hydroxyalkoxylated AON. In one embodiment, the hydroxyalkoxylated AON has improved cellular uptake than the non-hydroxyalkoxylated AON. In one embodiment, the hydroxyalkoxylated AON has improved trafficking than the non-hydroxyalkoxylated AON. In one embodiment, the hydroxyalkoxylated AON has improved immunogenicity than the non-hydroxyalkoxylated AON. In some embodiments, the hydroxyalkoxylated AON has increased exon skipping activity. In some embodiments, the hydroxyalkoxylated AON has increased exon skipping activity and production of a functional or a semi-functional dystrophin protein.

Exon skipping activity can be measured by analysing total RNA isolated from hydroxyalkoxylated AON/mixture-treated muscle cell cultures or muscle tissue by reverse transcriptase quantitative or digital droplet polymerase chain reaction (RT-qPCR or RT-ddPCR) using dystrophin gene-specific primers flanking the targeted exon (Spitali et al., FASEB J 2013, 27(12): 4909-4916, Verheul et al., 2016; PLoS ONE 11(9):e0162467). The ratio of shorter transcript fragments, representing transcripts in which the targeted exon is skipped, to the total of transcript products is assessed (calculated as percentage of exon skipping induced by an oligonucleotide). Shorter fragments may also be sequenced to determine the correctness and specificity of the targeted exon to be skipped.

In certain embodiments, RNA modulation activity may be an increase or decrease in an amount of a nucleic acid or protein. In certain embodiments, such activity may be a change in the ratio of splice variants of a nucleic acid or protein. Detection and/or measuring of antisense activity may be direct or indirect. In certain embodiments, antisense activity is assessed by observing a phenotypic change in a cell or animal.

Biodistribution and biostability can be at least in part determined by a validated sandwich hybridization assay adapted from Straarup et al., Nucl. Acids Res. 2010, 38(20):7100-7111. In one embodiment, plasma or homogenized tissue samples are incubated with a specific capture oligonucleotide probe complementary to part of the AON analyte. After separation, a DIG-labeled oligonucleotide probe is hybridized to the other part of the AON analyte, and quantitative detection follows using an anti-DIG antibody-linked peroxidase. Plasma oligonucleotide concentrations (μg/mL) are monitored over time to assess the peak concentration (Cₘₐₓ), time to peak concentration (Tₘₐₓ), area under the curve (AUC) and half-life. End of study tissue sample concentrations (μg/g tissue) are measured to assess tissue distribution. Non-compartmental pharmacokinetic analysis is performed using the Phoenix software package (WinNonlin module, version 6.4, Pharsight, Mountainview, Calif.).

Accordingly, in one embodiment, the hydroxyalkoxylated AON has an increased exon skipping activity than the non-hydroxyalkoxylated AON. In one embodiment, the hydroxyalkoxylated AON has acceptable or a decreased immunogenicity as compared to the non-hydroxyalkoxylated AON. In one embodiment, the hydroxyalkoxylated AON has a better biodistribution than the non-hydroxyalkoxylated AON. In one embodiment, the hydroxyalkoxylated AON has acceptable or improved RNA binding kinetics than the non-hydroxyalkoxylated AON. In one embodiment, the hydroxyalkoxylated AON has improved thermodynamic properties, such as an increased exon skipping activity, by comparison to (i) the corresponding non-hydroxyalkoxylated antisense oligonucleotide, and/or to (ii) the corresponding mixture of said antisense oligonucleotide and hydroxyalkoxy group(s) constituting said hydroxyalkoxylated AON, wherein said mixture differs only from the hydroxyalkoxylated AON in that the hydroxyalkoxy group(s) are not linked to said AON by for example a covalent linkage. In other embodiments, the hydroxyalkoxylated AON has an improved pK profile and/or reduced side effect profile (e.g., complement activation and/or liver enzyme inhibition) as compared to (i) the corresponding non-hydroxyalkoxylated antisense oligonucleotide, and/or to (ii) the corresponding mixture of said antisense oligonucleotide and hydroxyalkoxy group(s) constituting said hydroxyalkoxylated AON, wherein said mixture differs only from the hydroxyalkoxylated AON in that the hydroxyalkoxy group(s) are not linked to said AON by for example a covalent linkage. In some embodiments, said corresponding antisense oligonucleotide and said AON of the corresponding mixture have the same sequence and are modified in the same way as the AON of the hydroxyalkoxylated AON.

Hydroxyalkoxy Groups

In one embodiment, a hydroxyalkoxy group of the hydroxyalkoxylated AONs provided herein comprises or consists of an ethylene glycol monomer, ethylene glycol oligomer or ethylene glycol polymer (also known as polyethylene glycol, PEG).

In another embodiment, a hydroxyalkoxy group, or at least one hydroxyalkoxy group, or all hydroxyalkoxy groups, of the hydroxyalkoxylated AON provided herein comprises or consists of an ethylene glycol monomer, ethylene glycol oligomer or ethylene glycol polymer (also known as polyethylene glycol, PEG). In one embodiment, the linkage between the AON and a hydroxyalkoxy group of the hydroxyalkoxylated AON is covalent.

PEGylation, i.e., the attachment of (chemically activated) hydroxyalkoxy groups, including ethylene glycol monomers or chains, is well known to a person skilled in the art. PEGylation can be done at the —OH group of the 5′ terminal monomer and/or the 3′ terminal monomer of a nucleic acid. This can be done directly or through a spacer (e.g. aminoalkyl hydroxyalkoxy group), for example by click chemistry as known by the person skilled in the art.

Also encompassed is the use of modified PEGylation, wherein said (poly)ethylene glycol is chemically modified and/or contains a moiety attached thereto. In this way said hydroxyalkoxy group acquires an additional property as known in the art. For example, said hydroxyalkoxy group may become cleavable or fluorescent. An example of modified PEGylation includes but is not limited to cleavable PEGylation, wherein the linkage is a degradable (cleavable) linkage. Examples include linkages that are responsive to, for example, pH, light, temperature, reductive or oxidative environments, nucleophiles, synthetic reagents, enzymes, proteases, cathepsin, click-to-release reactions or (other) external stimuli.

In one embodiment, the hydroxyalkoxy group is an unmodified ethylene glycol monomer, ethylene glycol oligomer or ethylene glycol polymer. In another embodiment, the hydroxyalkoxy group is a modified ethylene glycol monomer, ethylene glycol oligomer or ethylene glycol polymer (e.g., modified PEG).

In one embodiment, a hydroxyalkoxy group of the hydroxyalkoxylated AON comprises or consists of 1, 2, 3, 4, 5, 6, 7, 8, 9, 10, 11, 12, 13, 14, 15, 16, 17, 18, 19 or 20 ethylene glycol monomers. In some embodiments, said hydroxyalkoxy group comprises or consists of 1 to 20, 1 to 16, 1 to 12, 1 to 8, 1 to 6, 2 to 16, 2 to 12, 2 to 8, 2 to 6, 3 to 12, 3 to 8 or 3 to 6 ethylene glycol monomers. In some embodiments, said hydroxyalkoxy group comprises or consists of 1, 2, 3, 4, 5, 6, 7, 8, 9, 10, 11 or 12 ethylene glycol monomers. In some embodiments, said hydroxyalkoxy group comprises or consists of 3, 4, 5 or 6 ethylene glycol monomers. In some embodiments, said hydroxyalkoxy group comprises or consists of 3 or 6 ethylene glycol monomers. In some embodiments, said hydroxyalkoxy group comprises or consists of 3 ethylene glycol monomers.

In one embodiment, said hydroxyalkoxy group is a diethylene glycol, triethylene glycol (TEG), tetraethylene glycol, pentaethylene glycol or hexaethylene glycol (HEG) group. In some embodiments, said hydroxyalkoxy group is TEG or HEG. In some embodiments, said hydroxyalkoxy group is TEG.

In the hydroxyalkoxylated AONs provided herein, the hydroxyalkoxy group is attached to the AON using methods well known to those of skill in the art. For example, the 5′- and/or 3′-terminal OH of the AON may be derivatized as a phosphoramidite, chloroformate, chloramidate or thiophosphoramidite, which is then reacted with the hydroxyalkoxy group (e.g., diethylene glycol, triethylene glycol (TEG), tetraethylene glycol, pentaethylene glycol or hexaethylene glycol (HEG)) under standard coupling conditions to provide the hydroxyalkoxylated AON. Alternatively, the OH of the hydroxyalkoxy group (e.g., diethylene glycol, triethylene glycol (TEG), tetraethylene glycol, pentaethylene glycol or hexaethylene glycol (HEG)) is derivatized as a phosphoramidite, chloroformate, chloramidate or thiophosphoramidite, which is then reacted with the 5′- and/or 3′-terminal OH of the AON under standard coupling conditions to provide the hydroxyalkoxylated AON.

In one embodiment, the hydroxyalkoxy group is attached to the AON through a phosphate linker (PO). In another embodiment, the hydroxyalkoxy group is a TEG group and the hydroxyalkoxylated AON is TEG-PO-AON (i.e., HO(CH₂CH₂O)₃—P(O)(OH)-AON), where the TEG-PO is attached to the 5′-OH of the AON. In another embodiment, the hydroxyalkoxy group is a TEG group and the hydroxyalkoxylated AON is TEG-PO-AON, where the TEG-PO is attached to the 3′-OH of the AON. In another embodiment, two TEG groups are attached to the AON, one at the 3′-OH and the other at the 5′-OH, and the hydroxyalkoxylated AON is (TEG-PO)₂-AON.

In another embodiment, the hydroxyalkoxy group is attached to the AON through a phosphorothioate linker (PS). In another embodiment, the hydroxyalkoxy group is a TEG group and the hydroxyalkoxylated AON is TEG-PS-AON (i.e., HO(CH₂CH₂O)₃—P(S)(OH)-AON), where the TEG-PS is attached to the 5′-OH of the AON. In another embodiment, the hydroxyalkoxy group is a TEG group and the hydroxyalkoxylated AON is TEG-PS-AON, where the TEG-PS is attached to the 3′-OH of the AON. In another embodiment, two TEG groups are attached to the AON, one at the 3′-OH and the other at the 5′-OH, and the hydroxyalkoxylated AON is (TEG-PS)₂-AON.

In another embodiment, two TEG groups are attached to the AON, one at the 3′-OH and the other at the 5′-OH, and the hydroxyalkoxylated AON is TEG-PO-AON-PS-TEG; where TEG-PO is at 5′ and TEG-PS is at 3′; or where TEG-PO is at 3′ and TEG-PS is at 5′.

Compositions

In another embodiment, provided herein is a composition comprising a hydroxyalkoxylated AON provided herein.

In one embodiment, said composition comprises at least one excipient, and/or said hydroxyalkoxylated AON comprises at least one conjugated ligand that may further aid in enhancing the targeting and/or delivery of said composition and/or said hydroxyalkoxylated AON to a tissue and/or cell and/or into a tissue and/or cell. A composition can comprise one or more than one hydroxyalkoxylated AON as described herein. In one embodiment, an excipient can be a distinct molecule, but it can also be covalently linked to the hydroxyalkoxylated AON. In the first case, an excipient can be a filler, such as starch. In the latter case, an excipient can for example be a targeting ligand that is linked to the oligonucleotide of the hydroxyalkoxylated AON.

In one embodiment, said composition is for use as a medicament. Said composition is therefore a pharmaceutical composition. The pharmaceutical composition usually comprises a pharmaceutically acceptable carrier, including diluents and/or excipients. In one embodiment, a composition comprises a hydroxyalkoxylated AON provided herein and optionally further comprises a pharmaceutically acceptable carrier, including a formulation, filler, preservative, solubilizer, diluent, excipient, salt, adjuvant and/or solvent. Such pharmaceutically acceptable filler, preservative, solubilizer, diluent, salt, adjuvant, solvent and/or excipient may for instance be found in Remington: The Science and Practice of Pharmacy, 20th Edition. Baltimore, Md.: Lippincott Williams & Wilkins, 2000. The hydroxyalkoxylated AON as described herein may possess at least one ionizable group. An ionizable group may be a base or acid, and may be charged or neutral. An ionizable group may be present as ion pair with an appropriate counterion that carries opposite charge(s). Non-limiting examples of cationic counterions are sodium, potassium, cesium, Tris, lithium, calcium, magnesium, trialkylammonium, triethylammonium, and tetraalkylammonium. Non-limiting examples of anionic counterions are chloride, bromide, iodide, lactate, mesylate, besylate, triflate, acetate, trifluoroacetate, dichloroacetate, tartrate, lactate, and citrate. Examples of counterions have been described (e.g. Kumar, Pharm. Technol. 2008, 3, 128).

A pharmaceutical composition may comprise an aid in enhancing the stability, solubility, absorption, bioavailability, activity, pharmacokinetics, pharmacodynamics, cellular uptake, and intracellular trafficking of said hydroxyalkoxylated AON, in particular an excipient capable of forming complexes, nanoparticles, microparticles, nanotubes, nanogels, hydrogels, poloxamers or pluronics, polymersomes, colloids, microbubbles, vesicles, micelles, lipoplexes, and/or liposomes. Examples of nanoparticles include polymeric nanoparticles, (mixed) metal nanoparticles, carbon nanoparticles, gold nanoparticles, magnetic nanoparticles, silica nanoparticles, lipid nanoparticles, sugar particles, protein nanoparticles and peptide nanoparticles. An example of the combination of nanoparticles and oligonucleotides is spherical nucleic acid (SNA), as in e.g. Barnaby et al. Cancer Treat. Res. 2015, 166, 23.

In one embodiment, a pharmaceutical composition comprises at least one excipient that may further aid in enhancing the targeting and/or delivery of said composition and/or said hydroxyalkoxylated AON to a tissue and/or a cell and/or into a tissue and/or a cell. In one embodiment, a tissue or cell is a muscle tissue or muscle cell.

Many of these excipients are known in the art (e.g., see Bruno, Advanced Drug Delivery Reviews 2011; 63: 1210). Non-limiting examples of these excipients include polymers (e.g. polyethyleneimine (PEI), polypropyleneimine (PPI), dextran derivatives, butylcyanoacrylate (PBCA), hexylcyanoacrylate (PHCA), poly(lactic-co-glycolic acid) (PLGA), polyamines (e.g. spermine, spermidine, putrescine, cadaverine), chitosan, poly(amido amines) (PAMAM), poly(ester amine), polyvinyl ether, polyvinyl pyrrolidone (PVP), polyethylene glycol (PEG) cyclodextrins, hyaluronic acid, colominic acid, and derivatives thereof), dendrimers (e.g. poly(amidoamine)), lipids {e.g. 1,2-dioleoyl-3-dimethylammonium propane (DODAP), dioleoyldimethylammonium chloride (DODAC), phosphatidylcholine derivatives (e.g 1,2-distearoyl-sn-glycero-3-phosphocholine (DSPC)), lyso-phosphatidylcholine derivaties (e.g. 1-stearoyl-2-lyso-sn-glycero-3-phosphocholine (S-LysoPC)), sphingomyeline, 2-{3-(Bis-(3-amino-propyl)-amino)-propylamino}-N-ditetracedyl carbamoyl methylacetamide (RPR209120), phosphoglycerol derivatives (e.g. 1,2-dipalmitoyl-sn-glycero-3-phosphoglycerol, sodium salt (DPPG-Na), phosphaticid acid derivatives (1,2-di stearoyl-sn-glycero-3-phosphaticid acid, sodium salt (DSPA)), phosphatidylethanolamine derivatives (e.g. dioleoyl-L-R-phosphatidylethanolamine (DOPE), 1,2-distearoyl-sn-glycero-3-phosphoethanolamine (DSPE),2-diphytanoyl-sn-glycero-3-phosphoethanolamine (DPhyPE)), N-(1-(2,3-dioleoyloxy)propyl)-N,N,N-trimethylammonium (DOTAP), N-(1-(2,3-dioleyloxy)propyl)-N,N,N-trimethylammonium (DOTMA), 1,3-di-oleoyloxy-2-(6-carboxy-spermyl)-propylamid (DOSPER), (1,2-dimyristyolxypropyl-3-dimethylhydroxy ethyl ammonium (DMRIE), (N1-cholesteryloxycarbonyl-3,7-diazanonane-1,9-diamine (CDAN), dimethyldioctadecylammonium bromide (DDAB), 1-palmitoyl-2-oleoyl-sn-glycerol-3-phosphocholine (POPC), (β-L-Arginyl-2,3-L-diaminopropionic acid-N-palmityl-N-olelyl-amide trihydrochloride (AtuFECT01), N,N-dimethyl-3-aminopropane derivatives (e.g., 1,2-distearoyloxy-N,N-dimethyl-3-aminopropane (DSDMA), 1,2-dioleyloxy-N,N-dimethyl-3-aminopropane (DoDMA), 1,2-Dilinoleyloxy-N,N-3-dimethylaminopropane (DLinDMA)), 2,2-dilinoleyl-4-dimethylaminomethyl (1,3)-dioxolane (DLin-K-DMA), phosphatidylserine derivatives (e.g., 1,2-dioleyl-sn-glycero-3-phospho-L-serine, sodium salt (DOPS)), proteins (e.g. albumin, gelatins, atellocollagen), and peptides (e.g. protamine, PepFects, NickFects, polyarginine, polylysine, CADY, MPG). Carbohydrates and carbohydrate clusters as described herein, when used as distinct compounds, are also suitable for use as a first type of excipient.

In one embodiment, a composition may comprise at least one excipient that comprises or contains a covalently linked group as described herein to enhance targeting and/or delivery of the composition and/or of the hydroxyalkoxylated AON to a tissue and/or cell and/or into a tissue and/or cell, as for example muscle tissue or muscle cell. The covalently linked group may display one or more different or identical ligands. Non-limiting examples of covalently linked group ligands are e.g. peptides, carbohydrates or mixtures of carbohydrates (Han et al., Nature Communications, 2016, doi:10.1038/ncomms10981; Cao et al., Mol. Ther. Nucleic Acids, 2016, doi:10.1038/mtna.2016.46), proteins, small molecules, antibodies, polymers and drugs. Non-limiting examples of carbohydrate hydroxyalkoxylated AON group ligands are glucose, mannose, galactose, maltose, fructose, N-acetylgalactosamine (GalNac), glucosamine, N-acetylglucosamine (GlcNAc), glucose-6-phosphate, mannose-6-phosphate, and maltotriose. Carbohydrates may be present in plurality, for example as end groups on dendritic or branched hydroxyalkoxy group moieties that link the carbohydrates to the component of the composition. A carbohydrate can also be comprised in a carbohydrate cluster portion, such as a GalNAc cluster portion. A carbohydrate cluster portion can comprise a targeting moiety and, optionally, a hydroxyalkoxylated AON hydroxyalkoxy group. In some embodiments, the carbohydrate cluster portion comprises 1, 2, 3, 4, 5, 6, or more GalNAc groups. Any of the excipients disclosed herein may be combined together into one single composition as described herein.

The skilled person may select, combine and/or adapt one or more of the above or other alternative excipients and delivery systems to formulate and deliver a hydroxyalkoxylated AON for use as described herein.

Such a pharmaceutical composition as described herein may be administered in an effective concentration at set times to an animal, or a mammal. In one embodiment, a mammal is a human being. In another embodiment, the effective concentration of a hydroxyalkoxylated AON or composition is from 0.01 nM to 1 μM. In another embodiment, the concentration used is from 0.05 to 500 nM, or from 0.1 to 500 nM, or from 0.02 to 500 nM, or from 0.05 to 500 nM, or from 1 to 200 nM. Such concentrations are exemplary only and not intended to limit this disclosure in any way. Those skilled in the art will be able to readily determine the effective concentration of a hydroxyalkoxylated AON provided herein using methods well known in the art.

A hydroxyalkoxylated AON or a composition as defined herein for use may be suitable for direct administration to a cell, tissue and/or an organ in vivo of individuals affected by or at risk of developing a disease or condition as identified herein, and may be administered directly in vivo, ex vivo or in vitro. Administration may be via topical, systemic and/or parenteral routes, for example intravenous, subcutaneous, intraperitoneal, intrathecal, intramuscular, ocular, nasal, urogenital, intradermal, dermal, enteral, intravitreal, intracavernous, intracerebral, intrathecal, epidural or oral route.

In one embodiment, such a pharmaceutical composition may be encapsulated in the form of an emulsion, suspension, pill, tablet, capsule or soft-gel for oral delivery, or in the form of aerosol or dry powder for delivery to the respiratory tract and lungs.

In another embodiment, said hydroxyalkoxylated AON may be used together with another compound already known to be used for the treatment of said disease. Such other compounds may be used for reducing inflammation, for reducing muscle tissue inflammation, and/or an adjunct compound for improving muscle fiber function, integrity and/or survival and/or improve, increase or restore cardiac function.

Non-limiting examples of such other compounds are a steroid, a (gluco)corticosteroid, steroid-like agent (e.g., vamorolone (VBP15)), epicatechin, an ACE inhibitor (e.g., perindopril), an HDAC inhibitor (e.g., givinostat), an angiotensin II type 1 receptor blocker (e.g., losartan), angiotensin peptide (1-7) (e.g., TXA127), a tumor necrosis factor-alpha (TNFα) inhibitor, a TGFβ inhibitor (e.g., decorin), a NF-κB inhibitor (e.g., edasalonexent (CAT-1004)), human recombinant biglycan, a source of mIGF-1, a myostatin inhibitor (e.g., PF-06252616 or RG6206), mannose-6-phosphate, an antioxidant (e.g., idebenone), an ion channel inhibitor, dantrolene, a protease inhibitor, a phosphodiesterase inhibitor (e.g., a PDE5 inhibitor, such as sildenafil or tadalafil), an anti-inflammatory and/or antifibrotic agent (e.g., HT-100), an utrophin modulator (e.g., ezutromid), metformin, creatine monohydrate (CrM), heparin, a granulocyte colony-stimulating factor (GCSF) (e.g., filgrastim), a connective tissue growth factor (CTGF/CCN2) inhibitor (e.g., FG-3019), a calcium modulator (e.g., AT-300), an androgen receptor modulator (e.g., DT-200), L-citrulline, and/or L-arginine. Such combined use may be a sequential use: each component is administered in a distinct fashion, perhaps as a distinct composition. Alternatively each component may be used together in a single composition.

Compounds that are comprised in a composition described herein can also be provided separately, for example to allow sequential administration of the active components of the composition. In such a case, the composition is a combination of compounds comprising at least a hydroxyalkoxylated AON provided herein with or without a covalently linked ligand, and at least one excipient, as described above.

Methods of Use

In another embodiment, provided herein is a method for preventing, treating, curing, ameliorating and/or delaying a condition or disease as defined herein in an individual, in a cell (e.g., a muscle cell), tissue (e.g., a muscle tissue) or organ of said individual. The method comprises administering a hydroxyalkoxylated AON or a pharmaceutical composition as described herein to said individual or a subject.

The method provided herein wherein a hydroxyalkoxylated AON or a pharmaceutical composition as defined herein may be suitable for administration to a cell (e.g., a muscle cell), tissue (e.g., muscle tissue) and/or an organ in vivo of individuals affected by any of the herein defined diseases, and may be administered in vivo, ex vivo or in vitro. In one embodiment, an individual or a subject is a mammal. In one embodiment, an individual or a subject is a human being. In some embodiments, a subject is not a human. Administration may be via topical, systemic and/or parenteral routes, for example intravenous, subcutaneous, nasal, ocular, intraperitoneal, intrathecal, intramuscular, intracavernous, urogenital, intradermal, dermal, enteral, intravitreal, intracerebral, intrathecal, epidural or oral route, as is known to those of skill in the art.

Dose ranges of a hydroxyalkoxylated AON or pharmaceutical composition described herein are designed on the basis of rising dose studies in clinical trials (in vivo use), the design and execution of which are well known to those of skill in the art. A hydroxyalkoxylated AON as defined herein may be used at a dose from 0.01 to 200 mg/kg or 0.05 to 100 mg/kg or 0.1 to 50 mg/kg or 0.1 to 20 mg/kg, or from 0.5 to 10 mg/kg.

The ranges of dose of a hydroxyalkoxylated AON or pharmaceutical composition as provided herein are exemplary concentrations or doses for in vitro or ex vivo uses and are not exclusive. As well known to those of skill in the art, depending on the identity of the hydroxyalkoxylated AON used, the target cell to be treated, the gene target and its expression levels, the medium used and the transfection and incubation conditions, the concentration or dose of the hydroxyalkoxylated AON used may further vary.

In one embodiment, provided is a method for preventing, treating, and/or delaying Duchenne Muscular Dystrophy (DMD), comprising administering to a subject a hydroxyalkoxylated AON provided herein, or a pharmaceutical composition as described herein.

In another embodiment, provided is a method for diagnosis wherein the hydroxyalkoxylated AON provided herein is provided with a radioactive label or fluorescent label.

In another embodiment, provided herein is a hydroxyalkoxylated AON or a composition as described herein for use as a medicament or part of therapy, or applications in which said hydroxyalkoxylated AON or composition exert their activity intracellularly.

In one embodiment, a hydroxyalkoxylated AON or composition provided herein is for use as a medicament or part of a therapy for preventing, delaying, curing, ameliorating and/or treating Duchenne Muscular Dystrophy (DMD).

In another embodiment, provided herein is the use of a hydroxyalkoxylated AON or a composition provided herein in the manufacture of a medicament. In one embodiment, said use of a hydroxyalkoxylated AON or a composition provided herein in the manufacture of a medicament is for preventing, delaying, curing, ameliorating and/or treating Duchenne Muscular Dystrophy (DMD).

In one embodiment, a hydroxyalkoxylated AON provided herein is for use as a medicament for treating a disease or condition disclosed herein through splice modulation, such as through exon skipping, or exon inclusion, both of which are forms of splice switching. In one embodiment, exon 51 of dystrophin pre-mRNA is skipped. In one embodiment, exon 51 of human dystrophin pre-mRNA is skipped. In one embodiment, a disease is Duchenne Muscular Dystrophy (DMD).

In one embodiment, a hydroxyalkoxylated AON provided herein targets an exonic splicing enhancer (ESE). ESE sequences facilitate the recognition of genuine splice sites by the spliceosome (Cartegni et al., Nat Rev Genet 2002; 3(4):285-98; and Cartegni et al., Nucleic Acids Res 2003; 31(13):3568-71). A subgroup of splicing factors, called the SR proteins, can bind to these ESEs and recruit other splicing factors, such as U1 and U2AF to splice sites. The binding sites of the four most abundant SR proteins (SF2/ASF, SC35, SRp40 and SRp55) have been analyzed in detail and these results are implemented in ESE-finder, a web source that predicts potential binding sites for these SR proteins (Cartegni et al., Nat Rev Genet 2002; 3(4):285-98; and Cartegni et al., Nucleic Acids Res 2003; 31(13):3568-71).

In one embodiment, the hydroxyalkoxylated AON as described herein consisting or consisting essentially of one antisense oligonucleotide and one or two hydroxyalkoxy groups (as described in the section “Hydroxyalkoxy Groups”), induces skipping of exon 51 of the dystrophin pre-mRNA. In another embodiment, the antisense oligonucleotide of the hydroxyalkoxylated AON induces single-exon skipping and the complementarity of its sequence with the region targeted of a given dystrophin exon is from 90 to 100%. In certain embodiments, the hydroxyalkoxylated AONs contain 1 or 2 mismatch(es) in an oligonucleotide of 20 nucleotides or 1 to 4 mismatches in an oligonucleotide of 40 nucleotides. In other embodiments, the hydroxyalkoxylated AONs contain 1, 2, 3, 4 or 5 mismatches in an oligonucleotide of 10 to 50 nucleotides. In one embodiment, 0, 1 or 2 mismatches are present in an oligonucleotide of 10 to 50 nucleotides. In another embodiment, in an oligonucleotide of 10 to 33 nucleotides, 0, 1, 2 or 3 mismatches are present, or, 0, 1 or 2 mismatches are present. In other embodiments, in an oligonucleotide of 16 to 22 nucleotides, 0, 1 or 2 mismatches are present, or 0 or 1 mismatch is present.

In one embodiment, the antisense oligonucleotide of the hydroxyalkoxylated AON provided herein induces dystrophin pre-mRNA splicing modulation, said pre-mRNA splicing modulation alters production or composition of protein, which comprises exon skipping or exon inclusion. In one embodiment, said pre-mRNA splicing modulation comprises exon skipping. This pre-mRNA splicing modulation can be used in the context of a therapeutic application as disclosed herein. Splicing of a pre-mRNA occurs via two sequential transesterification reactions involving an intronic branch point and a splice site of an adjacent intron.

The objective of pre-mRNA splicing modulation can be to alter production of protein, most often the protein the RNA codes for. This production can be altered through increase or decrease of the level of said production. This production can also be altered through alteration of the composition of the protein that is actually produced, for example when pre-mRNA splicing modulation results in inclusion or exclusion of one or more exons, and in a protein that has a different amino acid sequence.

In the case of DMD, dystrophin pre-mRNA splicing modulation can be applied to skip one or more specific exons in the dystrophin pre-mRNA in order to restore the open reading frame of the transcript and to induce the expression of a shorter but (more) functional dystrophin protein, with the ultimate goal to be able to interfere with the course of the disease. In one embodiment, provided is a hydroxyalkoxylated AON, wherein said hydroxyalkoxylated AON induces dystrophin pre-mRNA splicing modulation, wherein said dystrophin pre-mRNA splicing modulation alters production of protein that is related to Duchenne Muscular Dystrophy (DMD).

In one embodiment, a hydroxyalkoxylated AON as disclosed herein can be used for inducing exon-skipping in the dystrophin pre-mRNA in a cell, in an organ, in a tissue and/or in an individual. In one embodiment, a hydroxyalkoxylated AON can be used for skipping exon 51 of the dystrophin pre-mRNA.

Binding of dystrophin to actin and to the DGC or DAPC complex may be visualized by either co-immunoprecipitation using total protein extracts or immunofluorescence analysis of cross-sections using various antibodies reacting with the different members of the complex, from a control (non-DMD) biopsy of one from a muscle suspected to be dystrophic, pre- and/or post-treatment, as known to the skilled person.

Individuals or patients suffering from Duchenne muscular dystrophy typically have a mutation in the gene encoding dystrophin (the DMD or dystrophin gene) that prevents synthesis of the complete protein, i.e. a premature stop codon prevents the synthesis of the C-terminus. In Becker muscular dystrophy the dystrophin gene also comprises a mutation compared to the wild type but the mutation does typically not result in a premature stop codon and the C-terminus is typically synthesized. As a result a functional or semi-functional dystrophin protein is synthesized that has at least the same activity in kind as the wild type protein, although not necessarily the same amount of activity. The genome of a BMD patient typically encodes a dystrophin protein comprising the N terminal part (first 240 amino acids at the N terminus), a cysteine-rich domain (amino acid 3361 till 3685) and a C-terminal domain (last 325 amino acids at the C-terminus) but in the majority of cases its central rod shaped domain is shorter than the one of a wild type dystrophin (Monaco et al., Genomics 1988; 2: 90-95). Antisense oligonucleotide-induced exon skipping for the treatment of DMD is typically directed to overcome a premature stop in the pre-mRNA by skipping an exon, in one embodiment in the central rod-domain shaped domain, to correct the open reading frame and allow synthesis of remainder of the dystrophin protein including the C-terminus, albeit that the protein is somewhat smaller as a result of a smaller rod domain. In one embodiment, an individual having DMD and being treated by a hydroxyalkoxylated AON as defined herein will be provided a dystrophin which exhibits at least to some extent an activity of a wild type dystrophin. In one embodiment, if said individual is a Duchenne patient or is suspected to be a Duchenne patient, a functional or a semi-functional dystrophin is a dystrophin of an individual having BMD: typically said dystrophin is able to interact with both actin and the DGC or DAPC, but its central rod shaped domain may be shorter than the one of a wild type dystrophin (Monaco et al., Genomics 1988; 2: 90-95). The central rod domain of wild type dystrophin comprises 24 spectrin-like repeats. For example, a central rod shaped domain of a dystrophin as provided herein may comprise 5 to 23, 10 to 22 or 12 to 18 spectrin-like repeats as long as it can bind to actin and to DGC.

Alleviating one or more symptom(s) of Duchenne Muscular Dystrophy in an individual using a hydroxyalkoxylated AON described herein may be assessed by any of the following non-limiting assays: prolongation of time to loss of walking, improvement of muscle strength, improvement of the ability to lift weight, improvement of the time taken to rise from the floor, improvement in the nine-meter walking time, improvement in the time taken for four-stairs climbing, improvement of the leg function grade, improvement of the pulmonary function, improvement of cardiac function, or improvement of the quality of life. Each of these assays is known to the skilled person. As an example, the publication of Manzur et al. (Wiley publishers, 2008. The Cochrane collaboration), gives an extensive explanation of each of these assays. For each of these assays, as soon as a detectable improvement or prolongation of a parameter measured in an assay has been found, it will mean that one or more symptoms of Duchenne Muscular Dystrophy has been alleviated in an individual using a compound described herein. Detectable improvement or prolongation is a statistically significant improvement or prolongation as described in Hodgetts et al. (Neuromuscular Disorders 2006; 16: 591-602). Alternatively, the alleviation of one or more symptom(s) of Duchenne Muscular Dystrophy may be assessed by measuring an improvement of a muscle fiber function, integrity and/or survival. In one embodiment, one or more symptom(s) of a DMD patient is/are alleviated and/or one or more characteristic(s) of one or more muscle cells from a DMD patient is/are improved. Such symptoms or characteristics may be assessed at the cellular, tissue level or on the patient self.

An alleviation of one or more characteristics of a muscle cell from a patient may be assessed by any of the following assays on a myogenic cell or muscle cell from a patient: reduced calcium uptake by muscle cells, decreased collagen synthesis, altered morphology, altered lipid biosynthesis, decreased oxidative stress, and/or improved muscle fiber function, integrity, and/or survival. These parameters are usually assessed using immunofluorescence and/or histochemical analyses of cross sections of muscle biopsies.

The improvement of muscle fiber function, integrity and/or survival may be assessed using at least one of the following assays: a detectable decrease of creatine kinase in blood, a detectable decrease of necrosis of muscle fibers in a biopsy cross-section of a muscle suspected to be dystrophic, and/or a detectable increase of the homogeneity of the diameter of muscle fibers in a biopsy cross-section of a muscle suspected to be dystrophic. Each of these assays is known to the skilled person.

Creatine kinase may be detected in blood as described in Hodgetts et al. (Neuromuscular Disorders 2006; 16: 591-602). A detectable decrease in creatine kinase may mean a decrease of 5%, 10%, 20%, 30%, 40%, 50%, 60%, 70%, 80%, 90% or more compared to the concentration of creatine kinase in a same DMD patient before treatment.

A detectable decrease of necrosis of muscle fibers can be assessed in a muscle biopsy, for example, as described in Hodgetts et al. (Neuromuscular Disorders 2006; 16: 591-602), using biopsy cross-sections. A detectable decrease of necrosis may be a decrease of 5%, 10%, 20%, 30%, 40%, 50%, 60%, 70%, 80%, 90% or more of the area wherein necrosis has been identified using biopsy cross-sections. The decrease is measured by comparison to the necrosis as assessed in a same DMD patient before treatment.

A detectable increase of the homogeneity of the diameter of a muscle fiber can be assessed in a muscle biopsy cross-section, for example, as described in Hodgetts et al. (Neuromuscular Disorders 2006; 16: 591-602). The increase is measured by comparison to the homogeneity of the diameter of a muscle fiber in a same DMD patient before treatment.

In one embodiment, a hydroxyalkoxylated AON as described herein provides said individual with a functional or a semi-functional dystrophin protein, and is able to, for at least in part decrease the production of an aberrant dystrophin protein in said individual.

In one embodiment, providing an individual with a functional or a semi-functional dystrophin protein means an increase in the production of functional or semi-functional dystrophin protein as earlier defined herein. Increasing the production of functional or semi-functional dystrophin mRNA, or functional or semi-functional dystrophin protein, means a detectable increase or at least 5%, 10%, 20%, 30%, 40%, 50%, 60%, 70%, 80%, 90%, 100%, 120%, 140%, 160%, 180%, 200% or more compared to the initial amount of functional or semi-functional mRNA, or functional or semi-functional dystrophin protein, as detectable by RT-digital droplet PCR (mRNA) (Verheul et al., PLoS ONE 2016; 11(9): e0162467) or immunofluorescence (Beekman et al., PLoS ONE 2014; 9(9): e107494), western blot, or capillary Western immunoassay (Wes; Beekman et al., PLoS ONE 2018; 13(4): e0195850) analysis (protein). In one embodiment, said initial amount is the amount of functional or semifunctional mRNA, or functional or semi-functional dystrophin protein, at the onset of inducing exon-skipping in the dystrophin pre-mRNA in a cell, in an organ, in a tissue and/or in an individual using a hydroxyalkoxylated AON as described herein. Decreasing the production of an aberrant dystrophin mRNA, or aberrant dystrophin protein, means that 90%, 80%, 70%, 60%, 50%, 40%, 30%, 20%, 10%, 5% or less of the initial amount of aberrant dystrophin mRNA, or aberrant dystrophin protein, is still detectable by RT-digital droplet PCR (mRNA) or immunofluorescence, western blot, or capillary Western immunoassay (Wes) analysis (protein). In one embodiment, said initial amount is the amount of aberrant dystrophin mRNA, or aberrant dystrophin protein, at the onset of inducing exon-skipping in the dystrophin pre-mRNA in a cell, in an organ, in a tissue and/or in an individual using a hydroxyalkoxylated AON as described herein. An aberrant dystrophin mRNA or protein is also referred to herein as a less functional (compared to a wild type functional dystrophin protein as earlier defined herein) or a non-functional dystrophin mRNA or protein. A non-functional dystrophin protein is a dystrophin protein which is not able to bind actin and/or members of the DGC protein complex. A non-functional dystrophin protein or dystrophin mRNA does typically not have, or does not encode a dystrophin protein with an intact C-terminus of the protein. The detection of a functional or semi-functional dystrophin mRNA or protein may be done as for an aberrant dystrophin mRNA or protein.

Once a DMD patient is provided with a functional or a semi-functional dystrophin protein by administration of a hydroxyalkoxylated AON provided herein, at least part of the cause of DMD is taken away. Hence, it would then be expected that the symptoms of DMD are at least partly alleviated, or that the rate with which the symptoms worsen is decreased, resulting in a slower decline.

Overview of the Sequence Listing

The table below is provided to assist the skilled reader in interpreting the disclosure herein, including the Sequence Listing. The Descriptions below are not intended to limit this disclosure in any way.

| SEQ ID NO | Description |
|---|---|
| 1 | Human dystrophin protein |
| 2 | Exon 51 |
| 3 | ESE motif 1 of exon 51 |
| 4 | Reverse complement of SEQ ID NO: 3 |
| 5 | ESE motif 2 of exon 51 |
| 6 | Reverse complement of SEQ ID NO: 5 |
| 7 | Drisapersen/Suvodirsen (WVE-210201) |
| 8 | Eteplirsen |
| 9-404 | Unmodified antisense oligonucleotides |
| 405-800 | Hydroxyalkoxylated AONs: AONs (SEQ ID NO: 9-404) with a |
| | hydroxyalkoxy group (TEG) at 5′ terminal monomer of said AON |
| 801-1196 | Hydroxyalkoxylated AONs: AONs (SEQ ID NO: 9-404) with a |
| | hydroxyalkoxy group (TEG) at 3′ terminal monomer of said AON |
| 1197-1592 | Hydroxyalkoxylated AONs: AONs (SEQ ID NO: 9-404) with a |
| | hydroxyalkoxy group (TEG) at 5′ terminal monomer of said AON |
| | and a hydroxyalkoxy group (TEG) at 3′ terminal monomer of said |
| | AON |
| 1593-1988 | Antisense oligonucleotides represented by |
| | SEQ ID NO: 9-404, wherein all cytosine bases are 5- |
| | methylcytosine |
| 1989-3176 | Hydroxyalkoxylated AONs represented by SEQ ID NO: 405-1592, |
| | wherein all cytosine bases are 5-methylcytosine |
| 3177-4760 | Antisense oligonucleotides represented by SEQ ID NO: 9-404 and |
| | hydroxyalkoxylated AONs represented by SEQ ID NO: 405-1592, |
| | comprising a single BNA scaffold modification in the monomer at |
| | the 5′-terminus |
| 4761-6344 | Antisense oligonucleotides represented by SEQ ID NO: 9-404 and |
| | hydroxyalkoxylated AONs represented by SEQ ID NO: 405-1592, |
| | comprising a single BNA scaffold modification in the monomer at |
| | the 3′-terminus |
| 6345-7928 | Antisense oligonucleotides represented by SEQ ID NO: 9-404 and |
| | hydroxyalkoxylated AONs represented by SEQ ID NO: 405-1592, |
| | comprising two BNA scaffold modifications where one is in the |
| | monomer at the 5′-terminus and the other is in the monomer at the |
| | 3′-terminus |
| 7929-9512 | Antisense oligonucleotides represented by SEQ ID NO: 9-404 and |
| | hydroxyalkoxylated AONs represented by SEQ ID NO: 405-1592, |
| | comprising two BNA scaffold modifications, one in the monomer |
| | at the 5′-terminus and the other in the adjacent monomer |
| 9513-11096 | Antisense oligonucleotides represented by SEQ ID NO: 9-404 and |
| | hydroxyalkoxylated AONs represented by SEQ ID NO: 405-1592, |
| | comprising two BNA scaffold modifications, one in the monomer |
| | at the 3′-terminus and the other in the adjacent monomer |
| 11097-12680 | Antisense oligonucleotides represented by SEQ ID NO: 9-404 and |
| | hydroxyalkoxylated AONs represented by SEQ ID NO: 405-1592, |
| | comprising four BNA scaffold modifications, one in the monomer |
| | at the 5′-terminus, one in the monomer adjacent to the 5′-terminus, |
| | one in the monomer at the 3′-terminus and one in the monomer |
| | adjacent to the 3′-terminus |
| 12681-22184 | Antisense oligonucleotides and hydroxyalkoxylated AONs |
| | represented by SEQ ID NO: 3177-12680, wherein all cytosine |
| | bases are 5-methylcytosine |
| 22185-22232 | Antisense oligonucleotides and hydroxyalkoxylated AONs |
| | comprising an antisense oligonucleotide with BNA scaffold |
| | modifications at specific monomers, wherein the corresponding |
| | unmodified antisense oligonucleotide may be represented by SEQ |
| | ID NO: 11. |
| 22233-22292 | Antisense oligonucleotides and hydroxyalkoxylated AONs |
| | comprising an antisense oligonucleotide with BNA scaffold |
| | modifications at specific monomers, wherein the corresponding |
| | unmodified antisense oligonucleotide may be represented by SEQ |
| | ID NO: 9. |
| 22293-22332 | Antisense oligonucleotides and hydroxyalkoxylated AONs |
| | comprising an antisense oligonucleotide with BNA scaffold |
| | modifications at specific monomers, wherein the corresponding |
| | unmodified antisense oligonucleotide may be represented by SEQ |
| | ID NO: 10. |
| 22333-22480 | Antisense oligonucleotide and hydroxyalkoxylated AONs |
| | represented by SEQ ID NO: 22185-22332, wherein all cytosine |
| | bases are 5-methylcytosine. |
| 22481-22485 | PCR primers |
| 22486-22490 | Further antisense oligonucleotides and hydroxyalkoxylated AONs |

### EXAMPLES

The following examples are offered for illustrative purposes only, and are not intended to limit the scope of the present application in any way.

### Example 1

Materials and Methods

Hydroxyalkoxylated AON

All antisense oligonucleotides (AONs) (Table 1) consist of 2′-O-methyl RNA monomers linked by phosphorothioate backbone linkages and at least one LNA modification (SEQ ID NO: 22333), the same applies to the AON of the hydroxyalkoxylated AON represented by SEQ ID NO: 22345. The AONs were synthesized using commercially available solid phase synthesis (SPS) oligonucleotide synthesizers, such as an OP-10 synthesizer (GE/ÄKTA Oligopilot), a MerMade 12 Synthesizer (BioAutomation), or a Cytiva synthesizer. Standard phosphoramidite protocols were utilized. Chemical synthesis of the oligonucleotide via phosphoramidite chemistry involves sequential coupling of activated phosphoramidite monomers to an elongating polymer, which is covalently attached via the 3′ terminus to a solid support matrix. The hydroxyalkoxy group, such as TEG or HEG (hexaethylene) group, in the hydroxyalkoxylated AONs was introduced using the corresponding phosphoramidite, chloroformate, chloramidate or thiophosphoramidite building blocks and standard synthesis protocols. The AONs were deprotected and cleaved in a two-step sequence (DEA followed by conc. NH₄OH treatment), purified by anion-exchange chromatography, desalted by size exclusion chromatography and lyophilized. Mass spectrometry confirmed the identity of all AONs, and purity (determined by UPLC-UV) was found acceptable for all AONs (>80%).

TABLE 1

| | SEQ |
|---|---|
| | ID |
| Sequence (5′-3′) | NO |
| GGUAAGUUC*UGUC*C*AAGC* | 22333 |
| TEG-GGUAAGUUC*UGUC*C*AAGC* | 22345 |

A = adenosine;
G = guanine;
U = uracil;
T = thymine;
C* = 5-methylcytosine;
G, C* = LNA nucleotides;
TEG = tri-ethylene glycol group.

Mouse Experiment

This mouse experiment was carried out according to the National Institute of Health (NIH) guidelines for the care and use of laboratory animals. hDMDΔ52/mdx mice (Leiden University Medical Center, Veltrop et al., 2018; PLoS ONE 13 (2):e0193289) were randomized into groups (n=10) taking into account baseline weight and male-female distribution. Mice received 1× weekly an intravenous tail vein injection with a single antisense oligonucleotide represented by SEQ ID NO: 22333 (18 mg/kg), starting at 5-6 weeks of age for a total of 13 weeks. Mice received the hydroxyalkoxylated AON represented by SEQ ID NO: 22345, starting at 5-6 weeks of age for a total of 13 weeks. Ten days after the last AON injection the animals were sacrificed and tissue samples collected (after transcardial perfusion with PBS in order to remove blood from the tissues). Quadriceps muscle tissue samples were snap frozen and stored at −80° C.

RNA Isolation and cDNA Synthesis

Quadriceps muscle tissue samples were homogenized in 1 mL Nucleozol (Macherey Nagel) by grinding in a MagNa Lyser using Lysing Matrix D Tubes (MP Biomedicals). Total RNA was extracted from the homogenate based on the manufacturer's instructions. For cDNA synthesis, 1000 ng of total RNA was used as input. cDNA was generated in 20 μL reactions using random hexamer primers and GoScript Reverse Transcriptase and an incubation of 40 minutes at 50° C.

Digital Droplet PCR Analysis

Specific Taqman minor groove binder (MGB) assays were designed (using Primer Express 3.0.1 software; Applied Biosystems) to detect the dystrophin transcript products with and without exon 51 (Table 2) and purchased from Applied Biosystems. Digital droplet PCR analysis was performed on 2 or 4 μL of cDNA in a 20 μL reaction volume using an annealing/extension temperature of 60 C according to the manufacturer's instructions (BioRad). Data was presented as percentage exon skip (N₀ skipped/(N₀ skipped+N₀ non-skipped)*100).

TABLE 2

| | | | | SEQ |
|---|---|---|---|---|
| | Target | | | ID |
| Assay | Exons | Sequences | | NO |
| DMD_ | 47/52 | Forward | TGAAAATAAGCTCAAGCAGA | 22481 |
| 47-52 | | primer | CAAATC | |
| | | Reverse | GACGCCTCTGTTCCAAATCC | 22482 |
| | | primer | | |
| | | Probe | CAGTGGATAAAGGCAACA | 22483 |
| DMD_ | 51/52 | Forward | GTGATGGTGGGTGACCTTGAG | 22484 |
| 51-52.2 | | primer | | |
| | | Reverse | GACGCCTCTGTTCCAAATCC | 22482 |
| | | primer | | |
| | | Probe | CAAGCAGAAGGCAACAA | 22485 |

Protein Lysate Preparation

Frozen muscle tissue sections were homogenized in a Lysing Matrix D Tube (MP Biomedicals) with 200 μL Protein Lysis Buffer (15% SDS; 75 mM Tris-HCl pH 6.8; 5% β-Mercaptoethanol and a Protease Inhibitor Cocktail tablet (Roche/Sigma), using a MagNA Lyser (Roche). The supernatants were transferred to a new tube and supplemented with glycerol (final concentration 20%). Twenty-fold dilutions of these lysates were measured for total protein concentration using Pierce 660 nm Protein Assay Reagent (Thermo Scientific) and Ionic Detergent Compatibility Reagent (Thermo Scientific), according to the manufacturer's instructions.

Wes Analysis

Wes (ProteinSimple) analysis was performed according to the manufacturer's instructions, using a 66-440 kDa Separation Module (ProteinSimple SM-W008) and instrument default settings. The muscle lysates were diluted to 312.5 μg/mL, resulting in a final loading amount of 1.25 μg per well. The antibodies used were 1:50 mouse anti-dystrophin antibody Mandys106 (Glenn Morris) and 1:100 rabbit anti-vinculin antibody E1E9V (Cell signaling), together with Anti-Mouse and Anti-Rabbit Detection Modules (ProteinSimple DM-002 and DM-001). Electropherograms were inspected for proper peak detection and peak areas were converted to percentage of healthy hDMD control using calibration curves. Dystrophin levels were normalized to vinculin. The reported results are (dystrophin/vinculin) expressed as hDMD.

Results

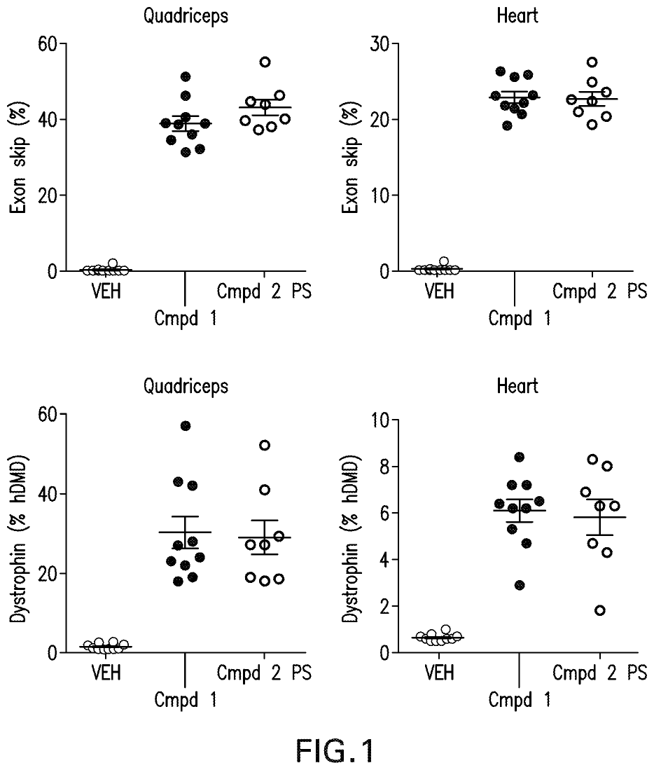

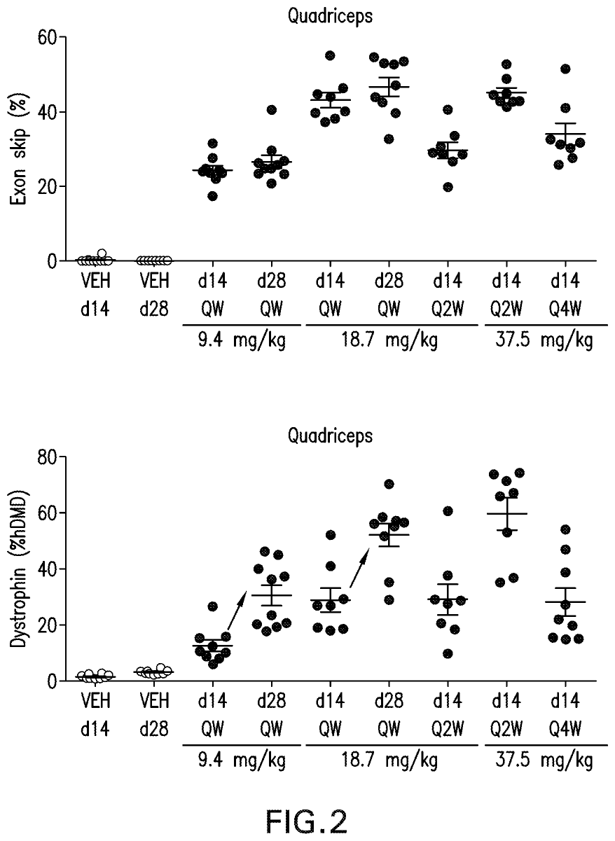

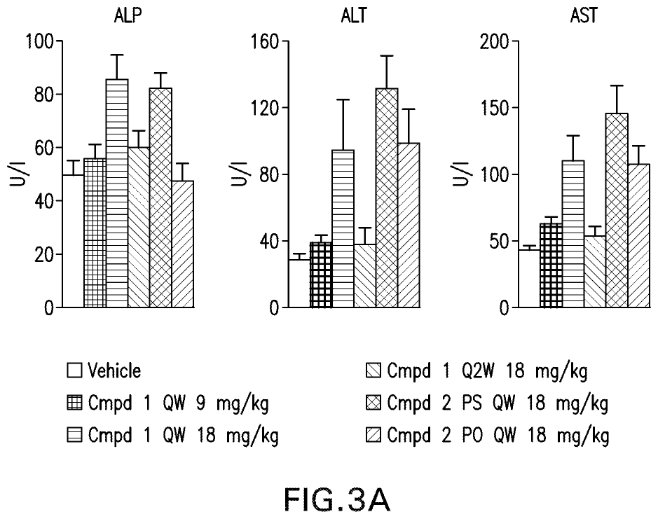

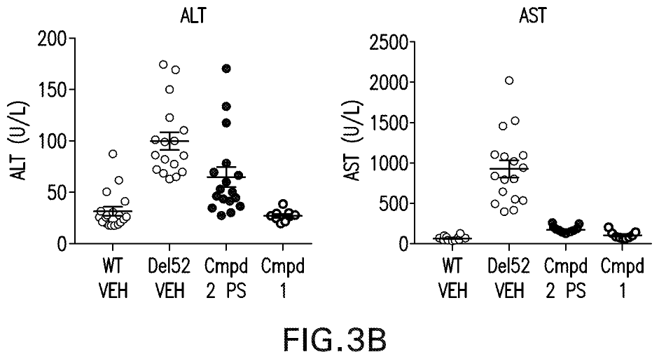

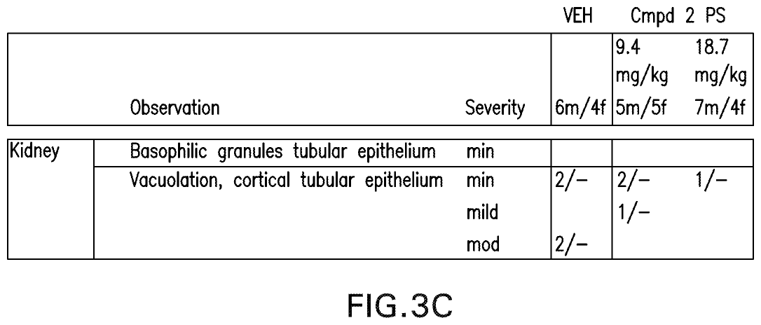

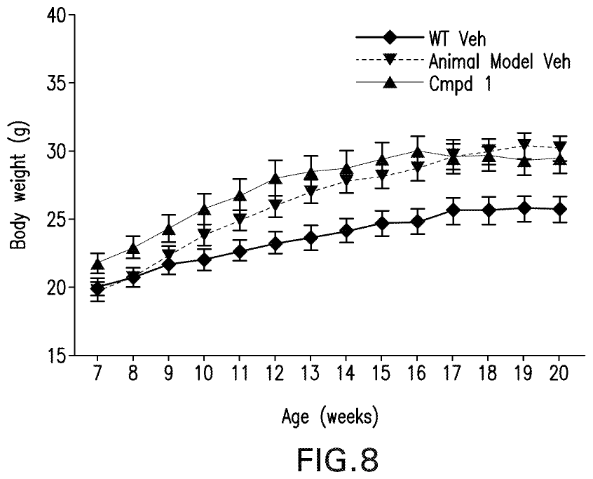

In this example, the exon skipping activity/efficiency and the production of a functional or semi-functional dystrophin protein were assessed for SEQ ID NOS: 22333 (Cmpd 1) and 22345 (Cmpd 2 PS). Exon skipping activity and production of a functional or semi-functional dystrophin protein is indicative of the bio-activity. Following a 13 week treatment (by weekly intravenous tail vein injections) with 18 mg/kg of the oligonucleotide with SEQ ID NO: 22333 (Cmpd 1), the dystrophin-deficient hDMDΔ52/mdx mice exhibited approximately a 30% increase in dystrophin expression in quadriceps biopsies (determined by WES analysis) when compared to mice treated with vehicle (FIG. 1). Similarly, when hDMDΔ52/mdx mice were treated with 18.7 mg/kg of the oligonucleotide hydroxyalkoxylated AON with SEQ ID NO: 22345 (Cmpd 2 PS) for 13 weeks by weekly intravenous tail vein injections, an approximately 30% increase in dystrophin expression in quadriceps was observed compared to mice treated with vehicle (FIG. 1). In these studies, both the oligonucleotide with SEQ ID NO: 22333 (Cmpd 1) and the oligonucleotide hydroxyalkoxylated AON with SEQ ID NO: 22345 (Cmpd 2 PS) exhibited approximately a 6% increase in dystrophin expression in heart at 14 days post-last-dose as compared to mice treated with vehicle, as determined by Western immunoassay (FIG. 1). In the case of SEQ ID NO: 22345 (Cmpd 2 PS), dystrophin continued to increase to 50% in quadriceps at 28-days post-last-dose, as compared to mice treated with vehicle (FIG. 2). When SEQ ID NO: 22333 (Cmpd 1) was evaluated for body weight effects, the drug-treated mice showed negligible body weight loss compared to the vehicle-treated mice (FIG. 8). SEQ ID NOS: 22333 (Cmpd 1) and 22345 (Cmpd 2 PS and Cmpd 2 PO) were evaluated for their impact on liver function by measuring alkaline phosphatase (“ALP”), alanine aminotransferase (“ALT”) and aspartate transaminase (“AST”) levels in healthy CD-1 mice. In healthy subjects, ALP, ALT and AST levels in the blood are low. If there is liver damage, more ALP, ALT or AST is released into the blood and levels will rise. The liver test showed ALP, ALT and AST levels in the drug-treated mice to be comparable to such levels the WT mice (with no disease) (FIG. 3A), and significantly lower ALT and AST levels than that in the vehicle-treated hDMDΔ52/mdx (diseased) mice (FIG. 3B). Dystrophin increases were accompanied by correction of neuronal nitric oxide synthase, and motor function improvement. Antisense oligonucleotide treatments with SEQ ID NO: 22345 (Cmpd 2 PS) (FIG. 3C) did not result in adverse renal effects, known to be a clinically translatable endpoint in mice. Absence of renal findings were, for the most part, confirmed in CD-1 mice (13 weekly doses at 9-18 mg/kg).

### Example 2

Cynomolgus Monkey Experiment

Eighteen normal male cynomolgus monkeys (˜3 kg at week one dose administration) were treated IV with the hydroxyalkoxylated AON with SEQ ID NO: 22345 (Cmpd 2 PO) or the AON having SEQ ID NO: 22333 (Cmpd 1) for 8-13 weeks at dose levels of 1-3 mg/kg as shown below (each Group had 6 monkeys). Each once-weekly IV dose given as a one-hour infusion via cephalic vein (chair restraint).

| | Dose | | | | Termination N | |
|---|---|---|---|---|---|---|
| | Article | | | | (Post-Final Dose) | |
| | (SEQ | | Dose | Dose | | 5 Weeks |
| | ID | IV Dose | Level | Volume | 1 Week | (Days 86 |
| Group | NO) | Regimen | (mg/kg) | (mL/kg) | (Day 57) | or 120) |
| 1 | 22333 | QW × 8 W | 1 | 2 | 3 | 3 (D86) |
| 2 | 22333 | QW × 8 W | 3 | 2 | 3 | 3 (D86) |
| 3 | 22345 | QW × 8 W/ | 1 | 2 | 3 (8 W) | 3 (13 W; |
| | PO | 13 W | | | | D120) |

In Groups 1 and 2, all animals were dosed QW×8W (i.e., on Days 1, 8, 15, 22, 29, 36, 43 and 50) for total of 8 doses before a 1- or 5-week recovery period (Days 57 & 86, respectively).

In Group 3, all animals were dosed QW×8W (i.e., on Days 1, 8, 15, 22, 29, 36, 43 and 50) for total of 8 doses. Subsequently,
(a) one cohort (n=3) had a 1-week recovery period (Day 57); and
(b) a second cohort (n=3) continued to receive five additional QW infusions (Days 57, 64, 71, 78 and 85) for total of 13 doses before a 5-week recovery period (Day 120).

Endpoint analysis (weekly BWs, observations and qualitative food consumption; clinical pathology (hematology, clinical chemistry & coagulation); extensive coagulation testing for dosing on Days 1, 22, 50 and 85; complement activation (Bb, C3a & sC5b-9); match extensive coagulation testing time points; urinalysis and urine chemistry; renal injury biomarkers; serum Cystatin C; urinary KIM-1, Clusterin, microalbumin & NAG; plasma and tissue TK; predose plasma collections through study; extensive plasma collections for dosing on Days 1, 22, 50 and 85; terminal plasma and tissue AON levels; tissue % exon skipping (liver, kidney, gastrocnemius); gross pathology and selected organ weights; histopathology (gall bladder, heart, kidney, liver, lung, lymph node (mesenteric), gastrocnemius, spleen, testis, kidney for possible Transmission Electron Microscopy (TEM))) demonstrated a lack of kidney or major organ toxicity.

Two complement analytes were tested to assess the level of complement activation. Bb is produced during alternative pathway complement activation, and C3a is produced during activation at the central part of complement. Bb and C3a have been demonstrated to be appropriate markers of activation in response to oligonucleotide-based therapeutics (Shen, L., Frazer-Abel, A., et al, J Pharmacol Exp Ther, 351:709-717, December 2014).

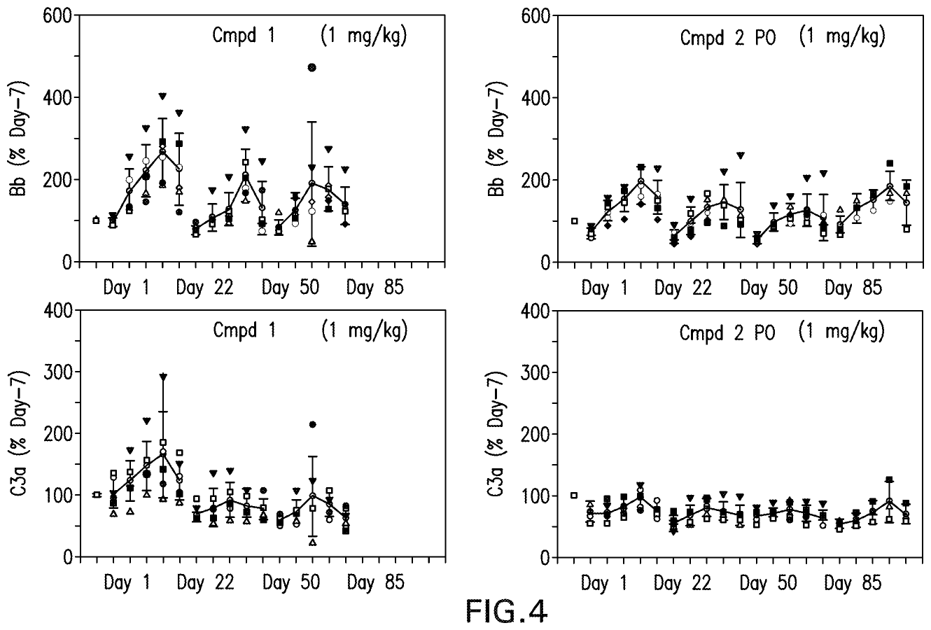

Figures of the data (FIG. 4) were calculated as percent Day −7. The data was presented this way to control for inter-animal differences in baseline complement levels.

The level of alternative pathway activation, as reflected by levels of the complement fragment Bb, increased after dosing on Day 1 for all treatment groups. The increase was greatest for animals treated with SEQ ID NO: 22333 (Cmpd 1) at 1 mg/kg with means exceeding two fold over baseline, in comparison to the 1 mg/kg SEQ ID NO: 22345 (Cmpd 2 PO)-treated animals which only increased to just below two fold. For SEQ ID NO: 22333 (Cmpd 1) the peak increase in measured Bb levels was reached on Day 1 at 6 hours post EOI. For SEQ ID NO: 22333 (Cmpd 1) dosed at 3 mg/kg (not shown), the pattern was consistent between 6 hours and 24 hours and, there was less of a decrease within 24 hours post EOI when compared to 1 mg/kg, indicating the alternative pathway activation observed in response to SEQ ID NO: 22333 (Cmpd 1) was not dose dependent. Additionally, the level of Bb measured on Day 22 and Day 50 trended to a lower group mean than that measured on first exposure on Day 1.

The second complement analyte measurement was C3a. C3a is produced when complement activation is strong enough to reach the central point of complement. Levels of C3a are important as it is an anaphylatoxin with downstream pro-inflammatory effects. Consistent with the measured Bb levels, the C3a levels measured in animals treated with SEQ ID NO: 22333 (Cmpd 1) were increased 1.6 fold increase over Day −7 levels. SEQ ID NO: 22345 (Cmpd 2 PO) at 1 mg/kg did not exhibit any increase in C3a across all days and timepoints measured.

The pattern of complement activation markers tested indicate the test article SEQ ID NO: 22333 (Cmpd 1) activates complement through the alternative pathway, impinging on the central point of complement but leading to an activation of the terminal pathway of less than three fold over pre-study levels, and was not consistent across the animals. This activation was transient, decreasing generally by 24 hours post EOI and returning to near baseline levels before the next once-weekly dose. The increases were generally not dose dependent and not additive, instead decreasing in level with repeat exposure. SEQ ID NO: 22345 (Cmpd 2 PO) only demonstrated mild activation of the alternative pathway with no clear increases downstream in the complement system. Further, the data at 22 and 50 days shows no overall accumulation of Bb and C3a.

Summary

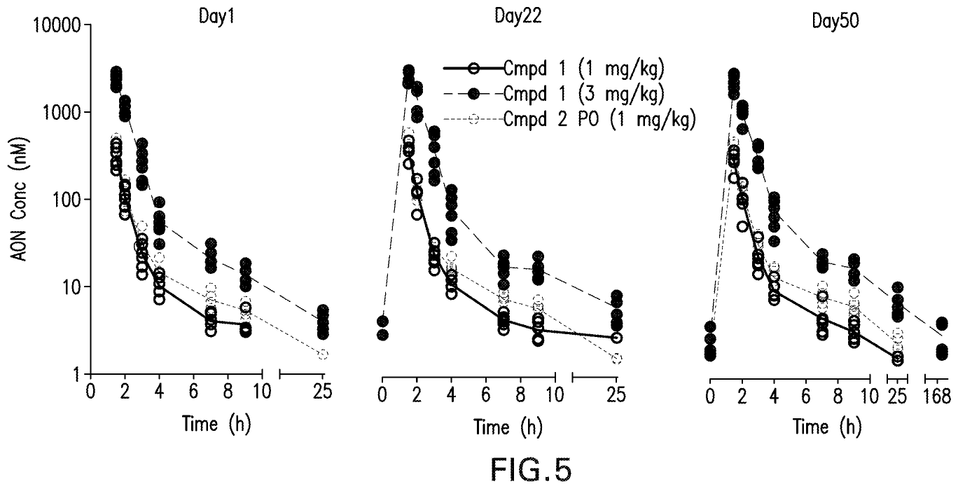

Antisense oligonucleotide-induced activation of the alternative complement pathway was transient and at sub-clinical levels, as measured by Bb and C3a analysis (FIG. 4); coagulation effects were also mild and transient (<5 sec prolongation of APTT). Plasma pK concentration for these AONs showed a dose proportional profile (FIG. 5).

### Example 3

Functional Motor Assay

Mice were tested at baseline and one week after last dose of hydroxyalkoxylated AON having SEQ ID NO: 22345 (Cmpd 2 PS) for walking behavioral tasks (Step, Stride, Stance, Swing analyses, Limb Coordination) according to the protocols below.

Open Field

Mice were tested in the Open field test at baseline, midpoint (after the 8th i.v. injection) and 1 week after last dose using Activity chambers (Med Associates Inc, St Albans, Vt.; 27×27×20.3 cm). Mice were brought to the experimental room for at least 30 min acclimation to the experimental room conditions prior to testing. Activity chambers were equipped with IR beams. Mice were placed in the center of the chamber and their behavior was recorded for 30 min. The following parameters were recorded: distance traveled, number of vertical rearings and average velocity.

Fine Motor Kinematic Analysis

Mice were tested in the MotoRater test at baseline, midpoint (after the 8th i.v. injection) and 1 week after last dose using walking behavioral tasks (Step, Stride, Stance, Swing analyses, Limb Coordination). On the day of testing, the mice were marked in appropriate points of body, such as joints of limbs and parts of tail to ease the data analysis process. The movement data was captured using a high speed camera (300 frames/second) from three different dimensions, from below and both sides. The captured videos of each mouse were first converted to SimiMotion software to track the marked points of body to have the raw data, i.e., the movement of the different body points in coordinates in relation to the ground, and each of the three dimensions were correlated. Different gait patterns and movements were analyzed using a custom made automated analysis system. The analyzed parameters included: 1) general gait pattern parameters (stride time and speed, step width, stance and swing time during a stride, interlimb coordination), 2) body posture and balance (toe clearance, iliac crest and hip height, hind limb protraction and retraction, tail position and movement), and 3) fine motor skills (swing speed during a stride, jerk metric during swing phase, angle ranges and deviations of different joints, vertical and horizontal head movement).

Principal Component Analysis

The gait parameters have always several (and complex) inter-correlations. For example, a shorter stride duration and longer step lengths lead to higher speed. Different “gait features”, which are manifested in sets of highly correlating parameters, can be identified using Principal Component Analysis (PCA).

Principal Component Analysis is a statistical tool to:
(a) compact the information in a multivariate data set,
(b) reveal correlations between original variables, and ultimately,
(c) create a small set of new and sensitive uncorrelated parameters, the principal components (PC).

PCA is a linear transformation based on principal component coefficients and eigenvectors. The transformed, new, uncorrelated variables are called the PC scores. The first principal component (PC) corresponds to such linear combination of data which has the largest possible variance. The second PC has again the largest possible variance of what is left when the proportion of the first PC is discarded, and so on for the rest of the PCs. The eigenvectors also reveal information about the internal structure of the data, i.e., mutually correlated parameters. Each PC score represent combined information of all the parameters which are emphasized in the corresponding PC.

For making the PC interpretation easier, several eigenvector rotation techniques exist. Rotation is a procedure in which the eigenvectors are manipulated to achieve simple structure, or the number of clearly non-zero elements of each eigenvector is optimized to be as low as reasonably possible. In this study, the orthogonality preserving, Varimax rotation procedure was used.

Finally, an overall gait analysis score based on PCA was determined. The score is based on differences between the hDMD del52/mdx and C57BL/6J vehicle groups in all the PC scores. Thus, the purpose of that score is to identify a disease model specific combination of original variables—a “fingerprint”—which characterizes the disease model in the best possible way and differentiates the two groups. After the “fingerprint”, or discriminant direction vector, has been determined, the overall gait analysis scores can be obtained by projecting the (normalized) parameter data of each individual mouse onto the discriminant direction vector. Ultimately, the overall kinematic effects of a pharmacological agent can be seen in a highly sensitive manner.

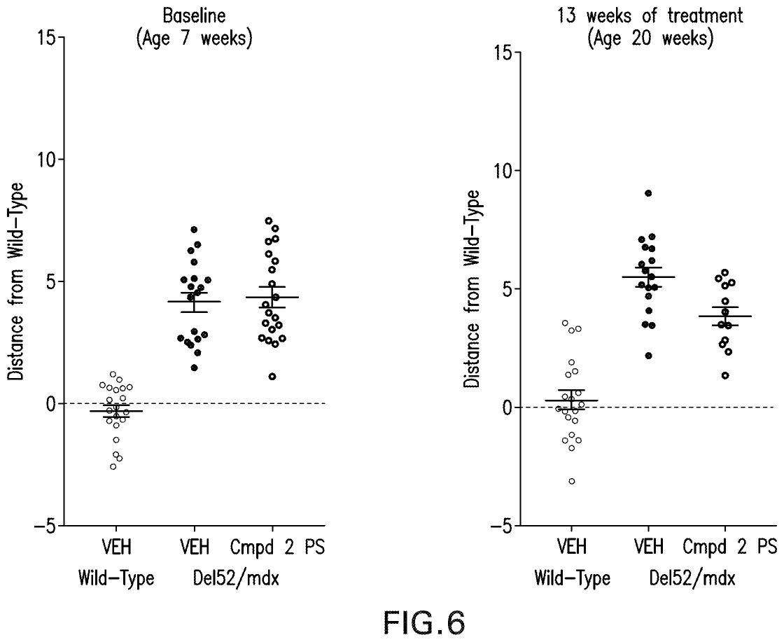

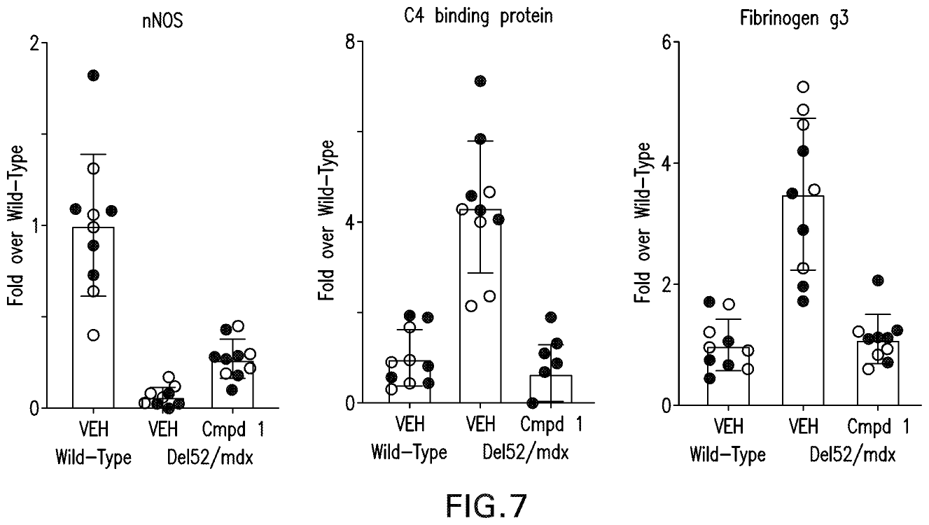

Results are shown in FIG. 6. Distance from WT=overall similarity of series of gait parameters to vehicle-treated wild-type control mice (see, e.g., DOI: 10.1089/nat.2019.0824).

### Example 4

Detection and Quantification of Dystrophin Protein and Biomarkers in Mouse Muscle Homogenate Using Liquid Chromatography (LC)-Parallel Reaction Monitoring (PRM)-Based Targeted Mass Spectrometry (MS)

The amount of dystrophin protein in the background of wildtype or DMD mouse muscle protein extract was determined using liquid chromatography (LC)-Parallel Reaction Monitoring (PRM)-based targeted mass spectrometry (MS).

Sample Preparation

All solvents were HPLC-grade from Sigma-Aldrich and all chemicals where not stated otherwise were obtained from Sigma-Aldrich.

Proteins were reduced and alkylated using Biognosys' reduction and alkylation buffers and digested overnight (constant μg protein/sample) with sequencing grade modified trypsin (Promega, cat #V5113) at a protein:protease ratio of 50:1. C18 cleanup for mass spectrometry was carried out on a C18 BioPureSPE Midi 96-well plate (The Nest Group) according to the manufacturer's instructions. Peptides were dried down to complete dryness using a SpeedVac system and re-dissolved in LC solvent A (1% acetonitrile in water with 0.1% formic acid (FA)) containing Biognosys' iRT-peptide mix (Ki-3002 Biognosys) for retention time calibration. Peptide concentration was measured using the mBCA assay kit (Pierce™).

Five stable isotope labeled standard (SIS) peptides to detect dystrophin proteins are listed in Table 3 (New England Peptides, the quality grade of the SIS peptides was ±10% quantification precision, >95% purity) were spiked into the final peptide samples at known concentrations.

TABLE 3
Five peptides representing mouse/human
dystrophin protein and their corresponding
AAA-quantified SIS peptides were measured
for absolute quantification

| Peptide | Protein ID | Species |
|---|---|---|
| TIMAGLQQTNSEK | P11531 | mouse |
| SPFPSQHLEAPEDK | P11532 | human |
| NIMAGLQQTNSEK | P11532 | human |
| LLDLLEGLTGQK | P11531; P11532 | mouse; human |
| IFLTEQPLEGLEK | P11531 | mouse |

Also, 3 SIS peptides to detect various biomarkers are listed in Table 4 (JPT, Berlin/Germany, the quality grade of the SIS peptides was SpikeTides_L, crude peptides (=not purified)) were spiked into the final peptide samples for relative quantification of the selected biomarker and control proteins.

TABLE 4
List of peptides representing mouse biomarker
proteins for relative quantification

| Entrez | Peptide | |
|---|---|---|
| ID | Sequence | Protein description |
| Q8VCM7 | TLEDILFR | Fibrinogen gamma |
| | | chain |
| Q9Z0J4 | VSKPPVIISDLIR | Nitric oxide |
| | | synthase, brain |
| P08607 | YECLPGYGR | C4b-binding protein |

SIS peptides had heavy labeled Arginine (Arg+10 Da) or Lysine (Lys+8 Da).

LC-PRM Measurements and Data Analysis

Peptides (1 μg per sample) were injected to an in-house packed C18 column (Dr. Maisch ReproSil-Pur 120 C18-AQ, 1.9 μm particle size, 120 Å pore size; 75 μm inner diameter, 50 cm length, New Objective) on a Thermo Scientific Easy nLC 1200 nano-liquid chromatography system. LC solvents were A: 1% acetonitrile in water with 0.1% FA; B: 15% water in acetonitrile with 0.1% FA. The LC gradient was 5-40% solvent B in 60 minutes followed by 40-100% B in 2 minutes and 100% B for 12 minutes (total gradient length was 74 minutes). LC-PRM runs for peptide quantification were carried out on a Thermo Scientific Q Exactive™ mass spectrometer equipped with a standard nano-electrospray source. Collision energies were 25 eV according to the vendor's specifications. An unscheduled run in PRM mode was performed before data acquisition for retention time calibration using Biognosys' iRT concept (Escher, Reiter et al., Proteomics 12 (2012), 1111-1121). The acquisition window was 7 minutes per peptide. Signal processing and data analysis were carried out using SpectroDive™ 9.0—Biognosys' software for multiplexed MRM/PRM data analysis based on mProphet (Reiter, Rinner et al., Nature Methods 8 (2011), 430-435). A Q-value filter of 1% was applied with additional manual inspection. The ratio of the peak areas (between those of the endogenous and those of the reference peptides) was used to determine the absolute levels of DMD proteins in the samples (results were expressed in fmol/μg peptides injected).

For biomarkers (measured with crude quality peptides), ratios of endogenous signals to the reference peptide signals were used as relative quantities.

### Example 5

SEQ ID NO: 22345 (Cmpd 2 PO) was synthesized on a 1 kg scale by solid phase synthesis (SPS) with a Cytiva synthesizer, using a support from Kinovate preloaded with Universal Linker (UnyLinker HL, NittoPhase). The sequence was synthesized by growing from the 3′ to 5′ end, one nucleoside at a time, by initially de-tritylating the 5′-position of the first nucleoside, using dichloroacetic acid in toluene, which was covalently attached to the solid support at the 3′ terminus. The phosphoroamidite containing the 2nd base was coupled in the presence of an acidic activator, benzylmercaptotetrazole (BMT) in acetonitrile, to build up the skeleton of the first dinucleotide. Thiolation of the resulting phosphite ester was executed with (N′-(3-thioxo-3H-1,2,4-dithiazol-5-yl)-N,N-dimethylmethanimidamide (DDTT) in pyridine/acetonitrile to form a thiophosphate ester. The phosphoramidites used in the synthesis were: 2′-O-4′-C-locked-5-Me-C(Bz) phosphoramidite, 2′-O-4′-C-locked-G (dmf) phosphoramidite, DMT-2′-O-Methyl-rA(Bz) phosphoramidite, DMT-triethyloxy-glycol phosphoramidite, 5-Me-DMT-2′-O-Me-C(Bz) CE phosphoramidite, DMT-2′-O-Methyl-rG(Ib) phosphoramidite and DMT-2′-O-Methyl-rU phosphoramidite. The last coupling was stabilized by iodine in pyridine/water. Capping any un-reacted 5′-hydroxyl groups was accomplished using reagents constituted from N-methylimidazole/Lutidine/acetonitrile and TAc₂O in acetonitrile.

Following the synthesis of the oligonucleotide, a diethylamine in acetonitrile solution was used to cleave the β-cyanoethyl protecting groups from the phosphorothioate backbone. Cleavage of the oligonucleotide from the resin and deprotection of the protecting groups on the nucleobases was executed with ethanolic ammonia, at 55° C. for several hours. The crude oligonucleotide solution was then separated from the solid support through various filtration steps. The filtered crude oligonucleotide solution was desalted against water and further concentrated. The oligonucleotide solution was further purified by anion exchange chromatography using Source Q as resin. A high concentration of sodium was used to elute the product from the column. The product was desalted against water by ultrafiltration, concentrated, and lyophilized to provide purified product (MS: 6518.76 Da).

### Example 6

SEQ ID NO: 22345 (Cmpd 2 PS): A procedure similar to that of Example 5 was followed to provide the hydroxyalkoxylated AON product (MS: 6534.40 Da).

### Example 7

Exon 51 Skipping

The AONs provided here were tested for exon 51 skipping in vitro according to assays known in the art (see, e.g., WO 2018/007475). Results are shown below.

| SEQ ID NO | % exon 51 skip |
|---|---|
| 22486 | 7.7 |
| 22487 | 7.6 |
| 22488 | 5.3 |
| 22489 | 3.4 |
| 22357 | 3.6 |
| 22369 | 4.0 |
| 22490 | 3.9 |

This disclosure is not to be limited in scope by the embodiments disclosed in the examples which are intended as single illustrations of individual aspects, and any equivalents are within the scope of this disclosure. Various modifications in addition to those shown and described herein will become apparent to those skilled in the art from the foregoing description. Such modifications are intended to fall within the scope of the appended claims.

Various references such as patents, patent applications, and publications are cited herein, the disclosures of which are hereby incorporated by reference herein in their entireties.

What is claimed is:

1. A hydroxyalkoxylated AON consisting of one antisense oligonucleotide and one or two hydroxyalkoxy groups, wherein said hydroxyalkoxy group comprises or consists of an ethylene glycol monomer, ethylene glycol oligomer or ethylene glycol polymer, wherein the antisense oligonucleotide is represented by a nucleotide sequence comprising or consisting of:
i) any one of SEQ ID NO: 9-404, or
ii) a fragment of any one of SEQ ID NO: 9-404, or
iii) any one of SEQ ID NO: 9-404 with 1, 2, 3, 4, or 5 additional nucleotides, or
iv) any one of SEQ ID NO: 9-404 with 1, 2, 3, 4, or 5 nucleotides missing from said SEQ ID NO, or
v) a nucleotide sequence which has at least 90% identity with any one of SEQ ID NO: 9-404.

2. The hydroxyalkoxylated AON of claim 1, wherein the antisense oligonucleotide consists of 2′-O-methyl RNA monomers linked by phosphorothioate backbone linkages.

3. The hydroxyalkoxylated AON of claim 1, wherein the hydroxyalkoxy group is diethylene glycol, triethylene glycol (TEG), tetraethylene glycol, pentaethylene glycol or hexaethylene glycol (HEG).

4. The hydroxyalkoxylated AON of claim 1, wherein the 5′ terminal monomer or the 3′ terminal monomer of said antisense oligonucleotide is linked to the hydroxyalkoxy group.

5. The hydroxyalkoxylated AON of claim 1, consisting of two hydroxyalkoxy groups, wherein the 5′ terminal monomer of said antisense oligonucleotide is linked to a first hydroxyalkoxy group and wherein the 3′ terminal monomer of said antisense oligonucleotide is linked to a second hydroxyalkoxy group, wherein the first and second hydroxyalkoxy groups comprise or consist of an ethylene glycol monomer, ethylene glycol oligomer or ethylene glycol polymer.

6. The hydroxyalkoxylated AON of claim 1, wherein the hydroxyalkoxy group is attached to the AON through a phosphate linker (PO).

7. The hydroxyalkoxylated AON of claim 1, wherein the hydroxyalkoxy group is attached to the AON through a phosphorothioate linker (PS).

8. The hydroxyalkoxylated AON of claim 1, wherein the antisense oligonucleotide has a length from 8 to 33 nucleotides.

9. The hydroxyalkoxylated AON of claim 1, wherein all cytosine bases of the antisense oligonucleotide are 5-methylcytosine bases.

10. The hydroxyalkoxylated AON of claim 1, wherein the antisense oligonucleotide comprises or consists of 1, 2, 3, 4, 5, 6, 7, 8 or 9 monomers that comprise a bicyclic nucleic acid (BNA) scaffold modification.

11. The hydroxyalkoxylated AON of claim 10, wherein the antisense oligonucleotide comprises or consists of BNA modifications as selected from the set consisting of:
(i) a single BNA scaffold modification in the monomer at the 5′-terminus,
(ii) a single BNA scaffold modification in the monomer at the 3′-terminus,
(iii) two BNA scaffold modifications where one is in the monomer at the 5′-terminus and the other is in the monomer at the 3′-terminus,
(iv) two BNA scaffold modifications, one in the monomer at the 5′-terminus and the other in the adjacent monomer,
(v) two BNA scaffold modifications, one in the monomer at the 3′-terminus and the other in the adjacent monomer, and
(vi) four BNA scaffold modifications, one in the monomer at the 5′-terminus, one in the monomer adjacent to the 5′-terminus, one in the monomer at the 3′-terminus and one in the monomer adjacent to the 3′-terminus;
optionally 1, 2, 3, 4 or 5 additional BNA scaffold modifications are present.

12. The hydroxyalkoxylated AON of claim 11, wherein the BNA modification is a bridged nucleic acid scaffold modification, or a locked nucleic acid (LNA) scaffold modification.

13. The hydroxyalkoxylated AON of claim 1, wherein all uracil bases of the antisense oligonucleotide are 5-methyluracil bases.

14. The hydroxyalkoxylated AON of claim 1 that has SEQ ID NO: 22345, 22357, 22369, 22488, 22489 or 22490.

15. The hydroxyalkoxylated AON of claim 14 that has SEQ ID NO: 22345.

16. A pharmaceutical composition, comprising the hydroxyalkoxylated AON of claim 1 and a pharmaceutically acceptable carrier.

17. A method of preventing, treating, and/or delaying Duchenne Muscular Dystrophy (DMD) in a subject, comprising administering to the subject the hydroxyalkoxylated AON of claim 1.

18. A method of inducing skipping of exon 51 of dystrophin pre-mRNA, comprising contacting the dystrophin pre-mRNA with the hydroxyalkoxylated AON of claim 1.

19. The hydroxyalkoxylated AON of claim 1, wherein the hydroxyalkoxy group is triethylene glycol (TEG).

20. The hydroxyalkoxylated AON of claim 1, wherein the 5′ terminal monomer of said antisense oligonucleotide is linked to the hydroxyalkoxy group.
